(19) United States
(12) Patent Application Publication
(10) Pub. No.: US 2026/0049133 A1
(43) Pub. Date: Feb. 19, 2026

(54) ANTIBODY-BASED MOLECULE OR COMPLEX, EXPRESSION SYSTEM AND PHARMACEUTICAL COMPOSITION THEREOF, AND METHODS THEREOF

(71) Applicants: Yong Yin, Riverside, CA (US); Yuan Zhou, Cupertino, CA (US)

(72) Inventors: Yong Yin, Riverside, CA (US); Yuan Zhou, Cupertino, CA (US)

(21) Appl. No.: 18/808,060

(22) Filed: Aug. 18, 2024

(51) Int. Cl. C07K 16/28; C07K 14/54; C07K 16/30

(52) U.S. Cl. CPC C07K 16/2803; C07K 14/5443; C07K 16/30; C07K 2317/31; C07K 2317/56; C07K 2317/92; C07K 2319/50

(57) ABSTRACT

The present invention provides an antibody-based molecule or complex capable of being selectively activated in a target cell or tissue and useful as therapeutics for treating various medical conditions. A moiety comprising a full-length antibody with a specific binding ability or a fragment thereof that maintains said specific binding ability is masked with a hetero dimer of peptides via protease cleavable linkers. The invention exhibits numerous technical merits such as improved selectivity and specificity of antibody and antibody-based therapeutics reaching their target within an intended location (e.g., a tumor cell) without non-specific binding to avoid on-target-off-site effects, among others.

### FIELD OF THE INVENTION

The present invention generally relates to an antibody-based molecule or complex useful as therapeutics for treating various medical conditions, an expression system thereof, a pharmaceutical composition thereof, and methods thereof. Although the invention will be illustrated, explained and exemplified by anti-PD-L1 antibody or Fv thereof masked by peptide hetero dimer of a mutant of interleukin 15 (IL-15) and IL-15 receptor alpha, it should be appreciated that the present invention can also be applied to other antibodies, antibody fragments, and antibody-based complexes, for example, anti-CD47 antibody, a conjugate EpCAM (Epithelial Cell Adhesion Molecule) antibody and cytotoxic drug, or a bispecific CD/47&EpCAM antibody, and the like, masked by peptide hetero dimer of a mutant of interleukin 15 (IL-15) and IL-15 receptor alpha or any other suitable peptide hetero dimers.

### STATEMENT REGARDING SEQUENCE LISTING

The instant application contains a Sequence Listing which has been submitted electronically in XML format and is hereby incorporated by reference in its entirety. Said Sequence Listing XML, created on Mar. 5, 2025, is named “V2 Full Antibody Patent Application for Yong and Kelly-sequence listing.xml” and is 69,632 bytes in size (on disc). The sequence listing contained in this .XML file is part of the specification and is hereby incorporated by reference herein in its entirety.

### BACKGROUND OF THE INVENTION

Antibodies are large, Y-shaped proteins belonging to the immunoglobulin superfamily which are used by the immune system to identify and neutralize antigens such as bacteria and viruses, including those that cause disease. Antibodies can recognize virtually any size antigen with diverse chemical compositions from molecules. Each antibody recognizes one or more specific antigens. Each tip of the “Y” of an antibody contains a paratope that specifically binds to one particular epitope on an antigen, allowing the two molecules to bind together with precision. Using this mechanism, antibodies can effectively “tag” a microbe or an infected cell for attack by other parts of the immune system, or can neutralize it directly (for example, by blocking a part of a virus that is essential for its invasion). To allow the immune system to recognize millions of different antigens, the antigen-binding sites at both tips of the antibody come in an equally wide variety. The rest of the antibody structure is relatively generic.

Antibody-based therapeutic agents are a rapidly evolving area of medical research and treatment. For example, monoclonal antibodies (mAbs) are used in treating various conditions, including cancers, autoimmune diseases, and infectious diseases. Examples include Rituximab (for lymphoma) and Trastuzumab (for breast cancer). Antibody-Drug Conjugates (ADCs) use an antibody combined with a cytotoxic drug, in which the antibody targets the cancer cells, delivering the drug directly to them with minimized damage to healthy cells. An example of ADCs is Brentuximab vedotin. Bispecific antibodies bind to two different antigens simultaneously. They can bring cancer cells and immune cells together to enhance the immune response against the cancer. Blinatumomab is an example used in treating certain types of leukemia. Checkpoint inhibitor antibodies block proteins that prevent the immune system from attacking cancer cells. By inhibiting these checkpoints, the immune system can better target and destroy cancer cells. Examples of checkpoint inhibitor antibodies include Pembrolizumab and Nivolumab. Recombinant antibodies produced through recombinant DNA technology can be designed to have specific properties, such as increased stability or reduced immunogenicity. Recombinant antibodies are used in various therapeutic applications, including cancer and autoimmune diseases. Antibody Fragments including Fab, Fv, F(ab′)2, Fab′, and scFv fragments are smaller than full antibodies and can penetrate tissues more easily. They are used in diagnostic and therapeutic applications.

One problem associated with current antibody-based therapeutic agents is the poor selectivity of site of action. Monoclonal antibodies and soluble fusion proteins are specific for binding to and neutralizing their intended target molecules (such as antigens and cell surface receptors). However, most target molecules are not specific to the disease site; rather, they may be present in cells or tissues other than the disease site. Accordingly, the therapeutic agent may act in these non-disease normal cells or tissues. This off-target action may result in unwanted side effects. Consequently, developing highly targeted antibody-based therapeutic agents is desirable.

Several approaches have been described for overcoming these off-target effects by engineering antibodies to have a cleavable linker attached to an inhibitory or masking domain that inhibits antibody binding. The linker can be designed to be cleaved by enzymes that are specific to certain tissues or pathologies, thus enabling the antibody to be preferentially activated in desired locations. Masking moieties can act by binding directly to the binding site of an antibody or can act indirectly via steric hindrance. Various masking moieties, linkers, protease sites and formats of assembly have been proposed. The extent of masking may vary between different formats as may the compatibility of masking moieties with expression, purification, conjugation, or pharmacokinetics of antibodies. There are two major categories of masking strategies developed in recent years for antibody and antibody derivative prodrug development. The first category is a physical/spatial hindrance-based approach. With this strategy, the mask fuses through a protease cleavable peptide with an antibody or one of antibody derivatives in its N terminal of either light chain or heavy chain or both to sterically interfere its binding an antigen, receptor or ligand. The mask could be a fragment of antibody, or autologous IgG hinge domain, Coiled-coil domain, non-antibody fragment, such as latency associated peptide and amino acid polymer chain. Another category is an affinity peptide-based approach. With this approach, a peptide binder, or a mutated antigen, or a modified receptor or a receptor fragment fuses to the N terminals of either light or heavy chain of an antibody through a protease cleavable peptide sequence to occupy the antigen, receptor or ligand binding site, and to exclude the antibody binding its target. Masked antibody, or masked antibody derivative has little or significantly reduced binding affinity to its ligand, receptor or antigen before the mask peptide or domain is removed from its masked antibody, or antibody derivative by a protease or proteases. It is well documented that the disease tissues, such as cancer, inflammatory tissues, have abnormally higher levels of proteases than normal tissues do. Those proteases are explored to remove the mask domain/peptide from masked antibody or antibody derivatives to activate the antibody or antibody derivatives function in desired loci.

However, there remains a need to improve antibody and antibody-based therapeutics selectivity and specificity to reach its target within an intended location (e.g., a tumor cell) without non-specific binding to avoid on-target-off-site effects.

### SUMMARY OF THE INVENTION

Advantageously, the present invention provides some solutions to meet the need. One aspect of the present invention provides an antibody-based molecule or complex capable of being selectively activated in a target cell or tissue. The molecule or complex includes a first mask peptide; a second mask peptide; and a masked moiety comprising a full-length antibody with a specific binding ability or a fragment thereof that maintains said specific binding ability. The first mask peptide is covalently connected to the masked moiety via at least a first protease cleavable linker. The second mask peptide is covalently connected to the masked moiety via at least a second protease cleavable linker. The first mask and the second mask peptide are non-covalently bound to each other forming a hetero dimer; and said hetero dimer inhibits said specific binding ability.

Another aspect of the invention provides an expression system used for producing the antibody-based molecule or complex as described above. The expression system may include (1) an isolated nucleic acid molecule encoding the antibody-based molecule or complex as described above or components thereof including the first mask peptide, the second mask peptide, the full-length antibody with a specific binding ability or a fragment thereof that maintains said specific binding ability, the first protease cleavable linker, and the second protease cleavable linker; (2) a vector comprising the isolated nucleic acid molecule of (1); and/or (3) a host cell transformed with the vector of (2).

Still another aspect of the invention provides a method of preparing the antibody-based molecule or complex as described above. The method may include the following steps: a) culturing a host cell comprising a vector that comprises an isolated nucleic acid molecule encoding the antibody-based molecule or complex as described above; and b) recovering or harvesting the antibody-based molecule or complex from the host cell culture.

Still another aspect of the invention provides a pharmaceutical composition comprising (i) the antibody-based molecule or complex as described above; an isolated nucleic acid molecule encoding the antibody-based molecule or complex; a vector comprising the isolated nucleic acid molecule; and/or a host cell transformed or transinfected with the vector; and (ii) a pharmaceutically acceptable carrier.

A further aspect of the invention provides a method of treating a disease or condition including cancer. The method may include administering a therapeutically effective amount of the antibody-based molecule or complex contained in or expressed by the pharmaceutical composition as described above to a subject in need thereof.

The above features and advantages and other features and advantages of the present invention are readily apparent from the following detailed description of the best modes for carrying out the invention when taken in connection with the accompanying drawings.

### BRIEF DESCRIPTION OF THE SEVERAL VIEWS OF THE DRAWINGS

The present invention is illustrated by way of example, and not by way of limitation, in the figures of the accompanying drawings and in which like reference numerals refer to similar elements. All the figures are schematic and generally only show parts which are necessary in order to elucidate the invention. For simplicity and clarity of illustration, elements shown in the figures and discussed below have not necessarily been drawn to scale. Well-known structures and devices are shown in simplified form, omitted, or merely suggested, in order to avoid unnecessarily obscuring the present invention.

FIG. 1 is a schematic diagram illustrating the structure of an antibody-based molecule or complex capable of being selectively activated in a target cell or tissue in accordance with an exemplary embodiment of the present invention.

FIG. 2 is a schematic diagram illustrating the structure of another antibody-based molecule or complex capable of being selectively activated in a target cell or tissue in accordance with an exemplary embodiment of the present invention.

FIG. 3 is a schematic diagram illustrating the structure of a molecule or complex based on a full-length antibody that is capable of being selectively activated in a target cell or tissue in accordance with an exemplary embodiment of the present invention.

FIG. 4 is a schematic diagram illustrating the structure of another molecule or complex based on a full-length antibody that is capable of being selectively activated in a target cell or tissue in accordance with an exemplary embodiment of the present invention.

FIG. 5 is a schematic diagram illustrating the structure of a molecule or complex based on an antibody fragment that is capable of being selectively activated in a target cell or tissue in accordance with an exemplary embodiment of the present invention.

FIG. 6 is a schematic diagram illustrating the structure of two molecules or complexes based on other antibody fragments that are capable of being selectively activated in a target cell or tissue in accordance with an exemplary embodiment of the present invention.

FIG. 7 shows a protease cleaving the cleavable linkers of an antibody-based molecule or complex in a target cell or tissue and selectively activating it in accordance with an exemplary embodiment of the present invention.

FIG. 8 shows the function of IL-15 mutant and IL-15 wild using human PBMC assay in accordance with an exemplary embodiment of the present invention.

FIG. 9 shows in vitro binding assay comparing the masked antibody to the un-masked counterpart antibody in accordance with an exemplary embodiment of the present invention.

FIG. 10 shows a cell-based function assay comparing the masked antibody to the un-masked antibody in accordance with an exemplary embodiment of the present invention.

FIG. 11 demonstrates that MMP2 and MMP9 protease substrate sites in the protease cleavable linker are cleavable and shows a dose-dependent manner in accordance with an exemplary embodiment of the present invention.

### DETAILED DESCRIPTION OF THE PREFERRED EMBODIMENT

In the following description, for the purposes of explanation, numerous specific details are set forth in order to provide a thorough understanding of the present invention. It is apparent, however, to one skilled in the art that the present invention may be practiced without these specific details or with an equivalent arrangement. Unless otherwise defined, all the technical terms used herein are used with the same meaning as commonly understood by a person skilled in the art in the field related to the present invention.

In the entire specification, when a part is referred to as “comprising” a component, it means that it may further include other components without excluding other components unless specifically described otherwise.

Where a numerical range is disclosed herein, unless otherwise specified, such range is continuous, inclusive of both the minimum and maximum values of the range as well as every value between such minimum and maximum values. Still further, where a range refers to integers, only the integers from the minimum value to and including the maximum value of such range are included. In addition, where multiple ranges are provided to describe a feature or characteristic, such ranges can be combined.

It is also to be understood that the terminology used herein is for the purpose of describing particular embodiments only and is not intended to limit the scope of the invention. For example, when an element is referred to as being “on”, “connected to”, or “coupled to” another element, it can be directly on, connected or coupled to the other element or intervening elements may be present. In contrast, when an element is referred to as being “directly on”, “directly connected to”, or “directly coupled to” another element, there are no intervening elements present.

Throughout the specification and claims, the following terms take the meanings explicitly associated herein, unless the context clearly dictates otherwise. The phrase “in one embodiment” does not necessarily refer to the same embodiment, although it may. Furthermore, the phrase “in another embodiment” does not necessarily refer to a different embodiment, although it may. Thus, as described below, various embodiments of the invention may be readily combined without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988 1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

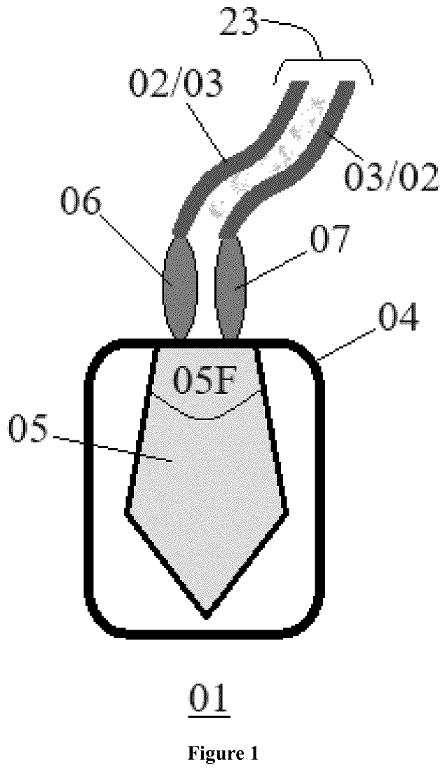

In various exemplary embodiments of the invention as illustrated in FIG. 1, an antibody-based molecule or complex 01 capable of being selectively activated in a target cell or tissue (not shown) includes a first mask peptide 02/03; a second mask peptide 03/02; and a masked moiety 04 comprising a full-length antibody 05 with a specific binding ability or an immunologically active fragment 05F thereof (hereinafter fragment 05F) that maintains the specific binding ability. Antibody 05 and fragment 05F may be recombinantly or synthetically produced.

The terms “antibody” and “immunoglobulin” refer broadly to any immunological binding agent, including polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like.

The full-length antibody 05 has a structure with two full-length light chains and two full-length heavy chains, and each light chain is linked to heavy chain by disulfide bond. Typically, a full-length antibody or a whole antibody consists of two antibody heavy chains and two antibody light chains. A heavy chain is a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in the case of an antibody of the subclass IgE. The light chain is a polypeptide consisting in N-terminal to C-terminal direction of an antibody light chain variable domain (VL), and an antibody light chain constant domain (CL), abbreviated as VL-CL. The antibody light chain constant domain (CL) can be K (kappa) or X (lambda). The antibody chains are linked together via inter-polypeptide disulfide bonds between the CL domain and the CH1 domain (i.e. between the light and heavy chain) and between the hinge regions of the antibody heavy chains.

Fragment 05F indicates a fragment retaining a antigen-binding function, and examples of fragment 05F include (i) Fab fragment consisting of light chain variable region (VL) and heavy chain variable region (VH), and light chain constant region (CL) and heavy chain 1st constant region (CH1); (ii) Fd fragment consisting of VH and CH1 domains; (iii) Fv fragment consisting of VL and VH domains of a monoclonal antibody; (iv) dAb fragment consisting of VH domain; (v) separated CDR region; (vi) F(ab′)2 fragment including two linked Fab fragments, as a divalent fragment; (vii) single chain Fv molecule (scFv) in which VH and VL domains are linked by a peptide linker to form an antigen binding site; (viii) bi-specific single chain Fv dimmer, (ix) multivalent or multi-specific diabody fragment prepared by gene fusion, and the like. The antibody 05 or immunologically active fragment 05F thereof may be selected from the group consisting of animal-derived antibody, chimeric antibody, humanized antibody, human antibody, and immunologically active fragment thereof.

The term “variable region” or “variable domain” refers to a portion of an antibody molecule that exhibits many variations on the sequence while performing a function that specifically binds to an antigen. In the variable region, there are complementarity determining regions CDR1, CDR2, and CDR3. A framework region (FR) portion exists between the CDRs to support a CDR ring. The “complementarity determining region” is a ring-shaped region involved in antigen recognition, and the specificity of the antibody for the antigen is determined as the sequence of this region changes. “Single chain fragment variable (scFv)” refers to a single chain antibody formed by expressing only a variable region of the antibody through genetic recombination and means an antibody in a single chain form in which the VH and VL regions of the antibody are linked to each other by a short peptide chain. The term “scFv” is intended to include an scFv fragment including an antigen-binding fragment.

An epitope is a site on an antigen to which an antibody binds. An epitope can be formed from contiguous amino acids or noncontiguous amino acids juxtaposed by tertiary folding of one or more proteins. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance.

Referring to FIG. 1 again, the first mask peptide 02/03 is covalently connected to the masked moiety 04 via at least a first protease cleavable linker 06. The second mask peptide 03/02 is covalently connected to the masked moiety 04 via at least a second protease cleavable linker 07. The first protease cleavable linker 07 and the second protease cleavable linker 07 may be different or the same, preferably they are the same. The first mask peptide 02/03 and the second mask peptide 03/02 are non-covalently bound to each other forming a hetero dimer 23; and the hetero dimer 23 inhibits the specific binding ability.

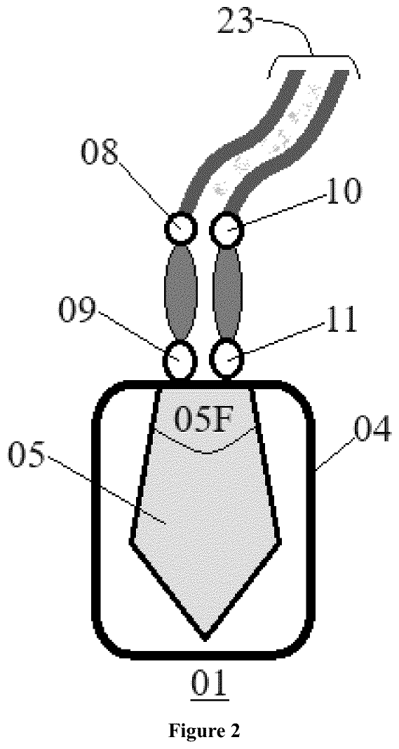

In some embodiments as illustrated in FIG. 2, the antibody-based molecule or complex 01 may include a spacer 08 between the first mask peptide 02/03 (C terminal) and the first protease cleavable linker 06 (N terminal) and/or a spacer 09 between the first protease cleavable linker 06 (C terminal) and the masked moiety 04 (N terminal). The antibody-based molecule or complex 01 may include a spacer 10 between the second mask peptide 03/02 (C terminal) and the second protease cleavable linker 07 (N terminal) and/or a spacer 11 between the second protease cleavable linker 07 (C terminal) and the masked moiety 04 (N terminal). The spacers (8, 9, 10, 11) may be, independently of each other, a short amino acid sequence (e.g. 2 to 5, or 5 to 10, or 10 to 30) such as (GS)n, (GGS)n, (GGGS)n, (GGGGS)n, (SG)n, (SGG)n, (SGGG)n, (SGGGG)n, (GSGS)n (GSG)n or any other artificial or natural short amino acid sequences. Throughout the specification, the conventional one-letter and three-letter codes for amino acids present in nature are used, as well as generally accepted three-letter codes for other amino acids. Additionally, the amino acids mentioned in abbreviation in the present invention are described according to the IUPAC-IUB Nomenclature: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

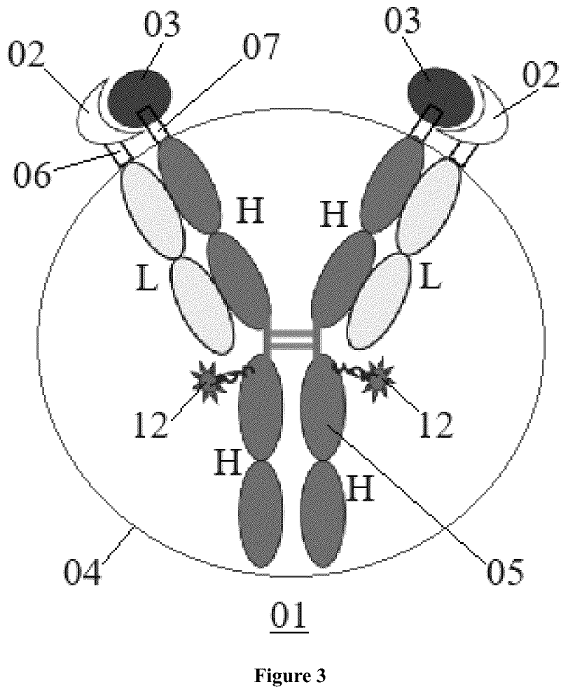

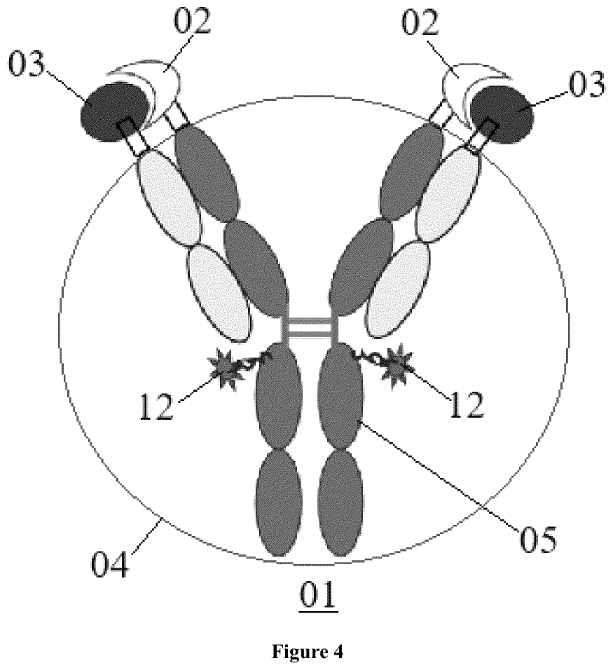

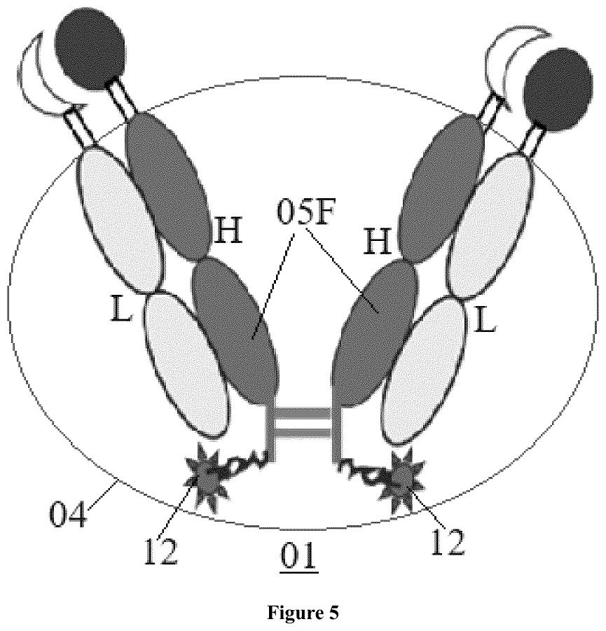

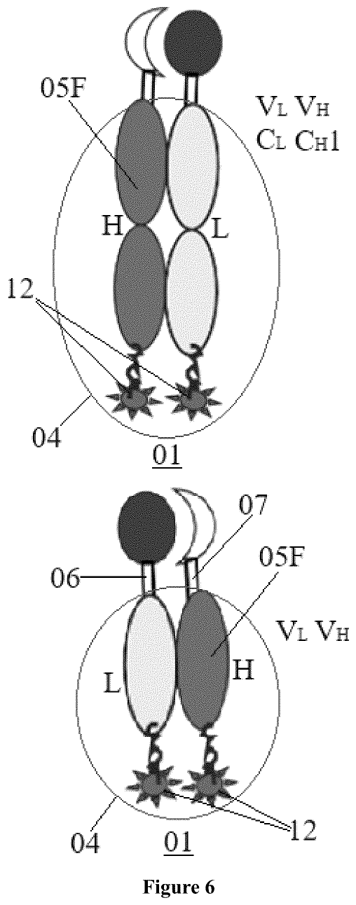

In some embodiments as illustrated in FIGS. 3 and 4, the masked moiety 04 in the antibody-based molecule or complex 01 may be an antibody toxin 04 or an immunotoxin 04, which may be a hybrid molecule that combines a full-length antibody 05 with a toxin 12. The antibody part 05 of the masked moiety 04 specific cells, such as cancer cells, because of the antibody 05's “specific binding ability,” while the toxin part 12 kills those cells once the antibody 05 binds to them. In some other embodiments as illustrated in FIGS. 5 and 6, the masked moiety 04 in the antibody-based molecule or complex 01 may be an antibody toxin 04 or an immunotoxin 04 that combines a toxin 12 with a fragment 05F of the full-length antibody 05 that maintains the specific binding ability of antibody 05. The antibody fragment part 05F of the masked moiety 04 specific cells, such as cancer cells, while the toxin part 12 kills those cells once the antibody fragment 05F binds to them. Examples of antibody fragment 05F include, but are not limited to, Fab (Fragment antigen-binding), Fv (Fragment variable), F(ab′)2 (Fragment antigen-binding, 2), Fab′ (Fragment antigen-binding, prime), and scFv (Single-chain variable fragment).

The full-length antibody 05 or the fragment thereof 05F may be a monoclonal antibody, bi-specific antibody, an antibody domain, or a domain antibody. Masked moiety 04 may include an antibody drug conjugate, an antibody toxin conjugate, or an antibody Fc fragment fusion. For example, masked moiety 04 may include an immune checkpoint inhibitor used in cancer immunotherapy such as anti-PD-L1 antibody or Fv thereof. Such an antibody-based molecule or complex 01 has dramatically lower affinity to human PD-L1 or lower IL-2 production in comparison to anti-PD-L1 antibody itself or Fv thereof. In other examples, masked moiety 04 may include anti-CD47 antibody, a conjugate EpCAM (Epithelial Cell Adhesion Molecule) antibody and cytotoxic drug, or a bispecific CD/47&EpCAM antibody.

As shown in FIGS. 3-6, the full-length antibody 05 or the fragment thereof 05F may include at least one pair of light chain L and heavy chain H (such as only one pair in FIG. 6). N terminal of the light chain L is covalently connected to the first mask peptide 02 (or the second mask peptide 03). N terminal of the heavy chain L is covalently connected to the second mask peptide 03 (or the first mask peptide 02). As shown in FIGS. 3-5, the full-length antibody 05 or the fragment thereof 05F may include two pairs of light chain L and heavy chain H. Each of N terminals of the light chains Ls is covalently connected to the first mask peptide 02 (or the second mask peptide 03). Each of N terminals of the heavy chains Hs is covalently connected to the second mask peptide 03 (or the first mask peptide 02).

In various exemplary embodiments, the first mask peptide and the second mask peptide (02, 03) are, independently of each other, wildtype peptides or mutants of wildtype peptides that have no detectable biological function in vitro assay and have no biological function in vivo. For example, the first mask peptide may be a mutant (or a variant) of wildtype interleukin 15 (IL-15) and the second mask peptide may be wildtype interleukin 15 receptor alpha. Interleukin-15 (IL-15) is a cytokine having a size of about 12 to 14 kD. IL-15 belongs to the family of the four-helix-bundle cytokines. The mature peptide of natural human interleukin-15 contains 114 amino acids, including 4 cysteine residues. Two pairs of intramolecular disulfide bonds respectively formed by the connection of Cys35 and Cys85 and the connection of Cys42 and Cys88 play an important role in maintaining the spatial conformation and biological activity of IL-15. IL-15 binds to its specific receptor, IL-15Rα, which is expressed on antigen-presenting dendritic cells, monocytes and macrophages. The a subunit of the IL-15 receptor has a high affinity for IL-15. Under physiological conditions, IL-15 mostly forms a complex (IL-15-Ra) with the a subunit, which enhances the affinity of IL-15 for the β chain subunit and the γ chain subunit of the receptor and activates T cells and NK cells.

A wild type is a phenotype, genotype, or gene that predominates in a natural population of organisms or strain of organisms in contrast to that of natural or laboratory mutant forms. For example, D8A mutation in the IL-15 sequence refers to the substitution of aspartic acid (D) at position 8 with alanine (A). This kind of mutation can affect the protein's structure and function, potentially altering its interaction with receptors or other molecules. In preferred embodiments of the invention, the mutant (or variant) of wildtype interleukin 15 has little or no binding to the IL-15 signaling receptors including IL-2 receptor beta (CD122) and the common gamma chain (CD132)/gamma (IL-2RBG), and/or it has no detectable activity to stimulate T cell activation, as compared to wildtype IL-15 or a wildtype IL-15 receptor alpha-IL-15 fusion polypeptide.

The mutant of IL-15 may include single mutation, double mutations, triple mutations, tetra mutations or four plus mutations. For example, the mutant of IL-15 may include substitutions of amino acid in its wildtype sequence of SEQ ID NO. 3 at the following positions: (a) D8, N65 and L69; (b) D8, N65 and Q101; (c) D8, N65 and Q108; or (d) D8, N65, Q101 and Q108; and may optionally further include one or more substitutions of amino acid at positions L69 and/or V104. In specific embodiments, the substitutions of amino acid are: D8S, D8A, or D8C; N65A, N65Q, N65K, or N65S; L69A, or L69R; Q101D, Q101A, or Q101S; V104P, or V104A; and Q108A, Q108D, or Q108S.

Each of cleavable linkers (06, 07) may be a peptide substrate cleavable by an enzyme. Operatively, the cleavable linker, upon being cleaved by the enzyme, allows for activation of the antibody or the antibody derivative 04/05/05F (such as antibody drug conjugate, bi-specific antibody, domain antibody or antibody-toxin fusion). Preferably, the cleavable linker is selected so that activation occurs at the desired site of action, which can be a site in or near the target cells (e.g., carcinoma cells) or tissues. For example, the cleavable linker is a peptide substrate specific for an enzyme that is specifically or highly expressed in the site of action, such that the cleavage rate of the cleavable linker in the target site is greater than that in sites other than the target site.

In various exemplary embodiments as illustrated in FIGS. 1-6, the first protease cleavable linker 06 and the second protease cleavable linker 07 may include, independently of each other, an amino acid sequence sensitive to (and cleaved by) one or multiple proteases or peptidase. The protease has an expression level at the target cell or tissue such as tumor region or inflammatory region (e.g. of a tumor tissue) that is higher compared to the respective expression level in a non-target cell/tissue for example a tumor- or inflammation-free region (e.g. of a corresponding normal tissue). For example, linkers 06 and 07 may be recognized by a serine protease, a cysteine protease, an aspartate protease, a threonine protease, a glutamic acid protease, a metalloproteinase, a gelatinase, an asparagine peptide lyase, cathepsins, a pepsin, a matriptase, a legumain, a matrix metalloprotease (MMP), a MMP1, a MMP2, a MMP3, a MMP7, a MMP8, a MMP9, a MMP10, a MMP11, a MMP12, a MMP13, a MMP14, an ADAMS, an ADAM10, an ADAM12, an urokinase plasminogen activator (uPA), an enterokinase, a prostate-specific target (PSA, hK3), an interleukin-1β converting enzyme, a thrombin, a FAP (FAP-α), a dipeptidyl peptidase, a type II transmembrane serine protease (TTSP), a neutrophil elastase, or any combinations thereof.

The term “expression” refers to the biological production of a product encoded by a coding sequence. In most cases, a polynucleotide (i.e., DNA) sequence, including the coding sequence, is transcribed to form a messenger-RNA (mRNA). The messenger-RNA is then translated to form a polypeptide product that has a relevant biological activity. The process of expression may involve further processing steps to the RNA product of transcription, such as splicing to remove introns, and/or post-translational processing of a polypeptide product.

Various exemplary embodiments of the invention provide an expression system used for producing the antibody-based molecule or complex as described above. The expression system may include (1) an isolated nucleic acid molecule encoding the antibody-based molecule or complex 01 or components thereof including the first mask peptide, the second mask peptide, the full-length antibody with a specific binding ability or a fragment thereof that maintains said specific binding ability, the first protease cleavable linker, and the second protease cleavable linker (hereinafter “the proteins of the present invention” or “the protein of the present invention”); (2) a vector comprising the isolated nucleic acid molecule of (1); and/or (3) a host cell transformed or transfected with the vector of (2). Also provided is a method of preparing the antibody-based molecule or complex as described above. The method may include the following steps: a) culturing a host cell comprising a vector that comprises an isolated nucleic acid molecule encoding the antibody-based molecule or complex as described above; and b) recovering or harvesting the antibody-based molecule or complex from the host cell culture.

The nucleic acid molecule may be isolated or recombinant and may include not only DNA and RNA in single-chain and two-chain form, but also corresponding complementary sequences. The isolated nucleic acid is a nucleic acid that has been isolated from surrounding genetic sequences present in the genome of a subject from which the nucleic acid was isolated, in the case of a nucleic acid isolated from a naturally occurring source. In the case of a nucleic acid synthesized enzymatically or chemically from a template, such as a PCR product, a cDNA molecule, or an oligonucleotide, the nucleic acid produced from such a procedure may be understood as an isolated nucleic acid molecule. The isolated nucleic acid molecule refers to a nucleic acid molecule in the form of a separate fragment or as a component of a larger nucleic acid construct. The nucleic acid is operably linked when placed in a functional relationship with another nucleic acid sequence. For example, DNA of a pre-sequence or secretory leader is expressed as a preprotein in the form before the polypeptide is secreted to be operably linked to DNA of the polypeptide, and a promoter or enhancer is operably linked to a coding sequence when affecting the transcription of the polypeptide sequence, or a ribosome binding domain is operably linked to a coding sequence when disposed to promote the translation. In general, the “operably linked” means that DNA sequences to be linked are located contiguously, and that the secretory leader exists contiguously in the same reading frame. However, the enhancer does not need to be contiguously located. The linkage is achieved by ligation at a convenient restriction enzyme site. When there is no such site, synthetic oligonucleotide adapters or linkers are used according to conventional methods.

In the isolated nucleic acid molecule encoding the proteins of the present invention due to the degeneracy of codons or in consideration of codons preferred in an organism in which the protein is to be expressed, it will be well understood to those skilled in the art that various modifications may be made in a coding region within a range without changing the amino acid sequence of the protein to be expressed from the coding region, various modifications or changes may be made within a range without affecting the expression of the gene even in parts other than the coding region, and such modified genes are also included within the scope of the present invention. That is, as long as the nucleic acid molecule of the present invention encodes a protein having equivalent activity thereto, one or more nucleobases may be mutated by substitution, deletion, insertion, or a combination thereof, which are also included in the scope of the present invention. The sequence of such a nucleic acid molecule may be single- or two-chain and may be a DNA molecule or an RNA (mRNA) molecule.

The isolated nucleic acid molecule encoding the proteins of the present invention may be inserted into an expression vector for protein expression. The expression vector generally includes a protein operably linked, i.e., functionally related with a control or regulatory sequence, a selectable marker, an optional fusion partner, and/or an additional element. In appropriate conditions, the proteins of the present invention may be produced by a method of inducing the protein expression by culturing a host cell transformed with a nucleic acid, preferably an expression vector containing an isolated nucleic acid molecule encoding the protein of the present invention. A variety of suitable host cells including mammalian cells, bacteria, insect cells, and yeast may be used, but are not limited thereto. Methods for introducing exogenous nucleic acids into host cells are known in the art and will vary depending on a host cell to be used. Preferably, it is possible to produce E. coli, which has high industrial value due to low production cost, as a host cell.

Vector is a vehicle into which a nucleic acid sequence may be inserted for introduction into a cell capable of replicating the nucleic acid sequence. The nucleic acid sequence may be exogenous or heterologous. Those skilled in the art may construct vectors by standard recombinant techniques. The vector of the present invention may include a plasmid vector, a cosmid vector, a bacteriophage vector, a viral vector, etc., but is not limited thereto. The suitable vector includes a signal sequence or a leader sequence for membrane targeting or secretion in addition to expression regulatory elements such as a promoter, an operator, an initiation codon, a termination codon, a polyadenylation signal, and an enhancer and may be variously produced according to a purpose. The promoter of the vector may be constitutive or inductive. Further, the vector may include a selectable marker for selecting a host cell including a vector and a replicable expression vector includes a replication origin. When constructing the vector, expression regulatory sequences such as a promoter, a terminator, and an enhancer, sequences for membrane targeting or secretion, etc. may be appropriately selected according to a type of host cell intended to produce the antibody and may be variously combined depending on a purpose.

Expression vector is a vector including a nucleic acid sequence encoding at least a portion of a gene product to be transcribed. In some cases, the RNA molecule is then translated into a protein, a polypeptide, or a peptide. The expression vector may include various regulatory sequences. In addition to regulatory sequences that regulate transcription and translation, vectors and expression vectors may also include nucleic acid sequences that serve other functions.

The term “host cell” includes a eukaryote and a prokaryote and refers to any transformable organism capable of replicating the vector or expressing a gene encoded by the vector. The host cell may be transfected or transformed by the vector, which means a process in which an exogenous nucleic acid molecule is delivered or introduced into the host cell. For example, the host cell may be a bacterial or animal cell, the animal cell line may be a CHO cell, an HEK cell or an NSO cell, and the bacteria may be E. coli.

After the recovering of the proteins of the present invention from the host cell culture, purifying the proteins may be further included. They may be isolated or purified by various methods known in the art. Standard purification methods include chromatographic techniques, electrophoresis, immunoprecipitation, dialysis, filtration, concentration, and chromatofocusing techniques. As is well known in the art, a variety of natural proteins such as bacterial proteins A, G, and L may bind to the protein of the present invention, and thus these natural proteins may be used for the purification of the protein of the present invention. Purification may often be enabled by using a particular fusion partner.

The vector of the present invention may be transformed into a suitable host cell, for example, E. coli or yeast cell, and the transformed host cell may be incubated to produce the proteins of the present invention in mass quantities. Incubation methods and media conditions suitable for a kind of host cell may be easily chosen from those known to those skilled in the art. The host cell may be a prokaryote such as E. coli or Bacillus subtilis. In addition, it may be a eukaryotic cell derived from yeast such as Saccharomyces cerevisiae, an insect cell, a plant cell, and an animal cell. More preferably, the animal cell may be an autologous or allogeneic animal cell. A transformant prepared through introduction into an autologous or allogeneic animal cell may be administered to a subject for use in cellular therapy against cancer. As for a method for introducing an expression vector into the host cell, it is possible to use any method if it is known to those skilled in the art. Other organisms, including transgenic (for example, genetically engineered) mice, or other mammals, may be used to produce the proteins of the present invention.

Various exemplary embodiments of the invention provide a pharmaceutical composition comprising the antibody-based molecule or complex 01 as described above; an isolated nucleic acid molecule encoding the antibody-based molecule or complex 01; a vector comprising the isolated nucleic acid molecule; and/or a host cell transformed or transinfected with the vector; and (ii) a pharmaceutically acceptable carrier. The pharmaceutical composition may include a second therapeutic, either biologics or small molecules/chemical compounds or both in the treatment of a disease or condition including cancer simultaneously or concurrently, for example, chemotherapeutic agents, immunomodulating agents, neuroactive agents, anti-inflammatory agents, anti-lipidemic agents, hormones, receptor agonists, receptor antagonists, anti-infective agents, anti-bacterial agents, anti-microbial agents, anti-fungal agents, proteins, peptides, nucleic acid molecules (including, but not limited to RNAs, DNAs, siRNAs, mRNAs, ribozymes, antisense molecules, etc.), hormones, cofactors, steroids, or any combination thereof.

Also provided in the present invention is a method of treating or ameliorating a disease or condition including cancer such as solid or liquid cancers and inflammation. The method may include administering a therapeutically effective amount of the antibody-based molecule or complex 01 contained in or expressed by the pharmaceutical composition as described above to a subject in need thereof.

Cancer refers to proliferative diseases, such as lymphomas, lymphocytic leukemias, lung cancer, non-small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers. Inflammation refers to arthritis, rheumatoid arthritis, pancreatitis, hepatitis, vasculitis, psoriasis, polymyositis, dermatomyositis, asthma, inflammatory asthma, autoimmune diseases (including e.g. lupus erythematosis, inflammatory arthritis), intestinal inflammatory diseases (including e.g. colitis, ulcerosa, inflammatory bowel disease, morbus crohn, celiac disease) and related diseases.

The term “treating or treatment” refers to all actions that alleviate or beneficially change the symptoms of the disease or condition by administering the pharmaceutical composition of the present invention. Those skilled in the art to which the present invention pertains will identify the exact criteria of a disease in which the pharmaceutical composition of the present invention is effective and determine the degree of alleviation, improvement and treatment.

The phrase “in need thereof” refers to a judgment made by a caregiver such as a physician or veterinarian that a patient requires (or will benefit in one or more ways) from treatment. Such judgment may made based on a variety of factors that are in the realm of a caregiver's expertise and may include the knowledge that the patient is ill as the result of a disease state that is treatable by the pharmaceutical compositions of the present invention.

The term “therapeutically effective amount” refers to an amount effective to prevent or treat the disease or condition, and the therapeutically effective amount of the pharmaceutical composition of the present invention may be determined by various factors, for example, administration method, target site, the patient's condition, and the like. Accordingly, the dosage when used in the human body should be determined in appropriate amounts in consideration of safety and efficacy. It is also possible to estimate the amount used in humans from the effective amount determined by animal experiments.

The pharmaceutical composition of the present invention may be administered in a pharmaceutically effective amount. The term “pharmaceutically effective amount” refers to an amount sufficient to treat the disease or condition at a reasonable benefit/risk ratio applicable for medical treatment and an amount that does not cause side effects. The level of an effective dosage may be determined by parameters including a health status of the patient, severity of the disease or condition, the activity of a drug, sensitivity to a drug, an administration method, administration time, an administration route and a release rate, duration of treatment, formulated or co-used drugs, and other parameters well known in medical fields. The pharmaceutical composition of the present invention may be administered as an individual therapeutic agent or in combination with other therapeutic agents. It may be administered sequentially or simultaneously with a conventional therapeutic agent or administered in a single- or multiple-dose regime. In consideration of all the above parameters, it is important to administer such a dose to obtain a maximum effect with a minimal amount without a side effect and the dose may be easily determined by those skilled in the art.

The pharmaceutical composition of the present invention may include carriers, diluents, excipients, or a combination of two or more thereof commonly used in biological formulations. The term “pharmaceutically acceptable” means that the composition is free of toxicity to cells or humans exposed to the composition. The carrier is not particularly limited as long as it is suitable for the delivery of the composition to the living body. For example, compounds, saline solutions, sterile water, Ringer's solution, buffered saline, dextrose solution, maltodextrin solution, glycerol, ethanol disclosed in Merck Index, 13th ed., Merck & Co. Inc. and one or more ingredients thereof may be mixed and used. If necessary, conventional additives such as antioxidants, buffers, and bacteriostatic agents may be added. The composition may also be prepared into dosage form for injection such as aqueous solution, suspension, or emulsion, tablet, capsule, powder or pill by additionally including diluents, dispersant, surfactant, binder and lubricant. Further, the composition may be formulated into a desirable form depending on targeting disease or ingredients thereof.

The pharmaceutical composition may be one or more formulations selected from the group consisting of oral formulations, external preparations, suppositories, sterile injectable solutions and sprays, and more preferably oral formations or injectable formulations.

The term “administration” means providing a predetermined substance to a subject or a patient by any appropriate method and may be administered orally or parenterally (for example, by applying in injectable formulations intravenously, subcutaneously, intraperitoneally, or topically). The dosage may vary depending on the patient's body weight, age, gender, health condition, diet, administration time, administration method, excretion rate, the severity of the disease, and the like. The liquid formulations for oral administration of the composition of the present invention include suspensions, oral liquids, emulsions, syrups and the like. In addition to water and liquid paraffin which are simple diluents commonly used, various excipients such as wetting agents, sweeteners, flavors, preservatives and the like may be included. Formulations for parenteral administration include sterile aqueous solutions, non-aqueous solvents, suspensions, emulsions, freeze-dried formulations, suppositories, and the like. The pharmaceutical composition of the present invention may be administered by any device capable of moving the active substance to target cells. The preferred administration method and formulations include intravenous, subcutaneous, intradermal, intramuscular, drip injections and the like. The injectable solution may be prepared using an aqueous solvent such as a physiological saline solution and Ringer's solution and a non-aqueous solvent such as a vegetable oil, a higher fatty acid ester (for example, ethyl oleate), an alcohol (for example, ethanol, benzyl alcohol, propylene glycol, glycerin, etc.) and may include pharmaceutical carriers such as stabilizer to prevent deterioration (for example, ascorbic acid, sodium hydrogen sulfite, sodium pyrosulfite, BHA, tocopherol, EDTA, etc.), an emulsifier, a buffer for pH control, preservatives for inhibition of microbial growth (for example, phenylmercuric nitrate, thimerosal, benzalkonium chloride, phenol, cresol, benzyl alcohol, etc.).

The term “subject” means an organism, including mammals such as primates, to which treatment with the pharmaceutical compositions of the present invention can be provided. Mammalian species that can benefit from the disclosed methods of treatment include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

The pharmaceutical composition of the present invention may further include a pharmaceutically acceptable additive, which is exemplified by starch, gelatinized starch, microcrystalline cellulose, milk sugar, povidone, colloidal silicon dioxide, calcium hydrogen phosphate, lactose, mannitol, taffy, Arabia rubber, pregelatinized starch, corn starch, cellulose powder, hydroxypropyl cellulose, Opadry, sodium starch glycolate, carnauba wax, synthetic aluminum silicate, stearic acid, magnesium stearate, aluminum stearate, calcium stearate, white sugar, dextrose, sorbitol, talc, etc. The pharmaceutically acceptable additive of the present invention is preferably added to the composition in an amount of 0.1 to 90 parts by weight but is not limited thereto.

Exemplary embodiments provide peptide variants of interleukin 15 and interleukin 15 receptor alpha (02, 03) as mask or shield block of an antibody (05 or 05F) or an antibody-based molecule 04. When they fuse to the N terminals of both light and heavy chains of an antibody (05 or 05F), or antibody-based therapeutics 04 through protease cleavable linker (06, 07), can prevent the antibody or antibody-based therapeutics from binding its partner. When the two hetero peptides (02, 03) are removed by protease cleavage of the protease cleavable linker (06, 07), the binding and function of the antibody (05 or 05F) or antibody-based therapeutics 04 will be restored.

Masked antibody or antibody derivative 01 is in an inactive form, which comprises: (i) the first moiety 23: mask domains A and B (02, 03), fused respectively to the N-terminals of the light chain L and the heavy chain H of an antibody or antibody derivatives (05 or 05F); (ii) the second moiety 04: target antibody or antibody derivative (05, 05F, 04) that is sterically occluded from binding a target by its N terminal mask domains; and (iii) a protease cleavable peptide linker (06, 07) connecting the first moiety 23 (mask domains 02 & 03) and the second moiety 04 (target antibody or antibody derivatives' light chain L and heavy chain H). Upon a protease cleavage of the cleavable peptide linker (06, 07), the target antibody or antibody derivative (05, 05F, 04) is activated after the mask domains 23 ae released, and the antibody binding domains are exposed to bind its target.

An exemplary masked antibody molecule 01 contains the first domain A and domain B (02, 03) tethered to a protease cleavable peptide linker (06, 07), the second domain is an anti-target antibody (05, 05F or 04, e.g. specific target on the tumor). The anti-target antibody (05, 05F, 04) is sterically occluded from binding its target on the tumor.

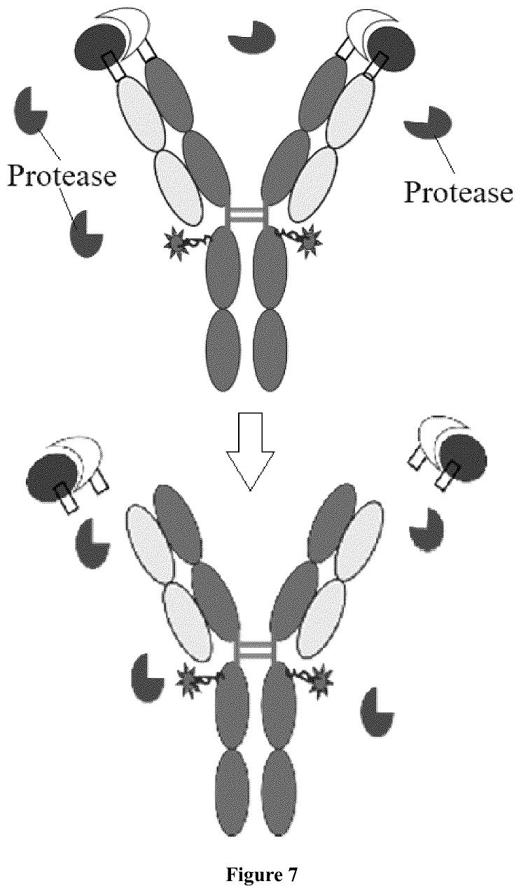

In treating or ameliorating a disease or condition including cancer such as solid or liquid cancers, a therapeutically effective amount of the antibody-based molecule or complex 01 contained in the pharmaceutical composition as described above is administered to a subject in need thereof, and then the antibody-based molecule or complex 01 is conditionally activated. For example, FIG. 7 illustrates a possible mechanism of conditional activation of a masked antibody-drug conjugate 01 by abnormal high level of proteases in tumor microenvironment.

Peptide sequences (02, 03) named “mask” can block the antibody or antibody-based therapeutics 04 from binding its antigen, receptor or ligand both in vitro and in vivo. The mask domains (02, 03) in the inactive form with fused antibody or antibody-based therapeutics 04 may be in close proximity to the binding sequence of an antibody, antibody-based therapeutics 04 wherein the binding sequence is specific for the target. In some embodiments, the target comprises a tumor antigen. In some embodiments, the tumor antigen comprises at least one of: Ep-CAM, EGFR, HER-2, HER-3, c-Met, FOLR, PSMA, CD38, BCMA, and CEA, B7-H3, Cadherin-6, CD19, CD20, CD22, CD40, CD44, CD52, CD56, CD70, CD71, CD74, FGFR2, FGFR3, GPC3, Mucd, Mucl6, NaPi2b, Nectin-4, P-cadherin, ROR1, TIM1, or Trop2.

In some exemplified embodiments, heterodimeric peptides (02, 03) consisting of domain A and B, as blocking/shielding entity or simply “mask”, are fused respectively to the N terminals' light and heavy chains of an antibody or an antibody derivative 04/05/05F (such as antibody drug conjugate, bi-specific antibody, domain antibody or antibody-toxin fusion) to prevent the antibody and the antibody derivative from binding to their target, such as receptor, ligand and antigen.

The two hetero peptides (02, 03) may be called domain A and domain B and can interact each other to form a heterodimer 23 as shown in FIG. 1. Domain A and domain B peptides (02, 03) fuse respectively to the N terminals of an antibody light chain and heavy chain through a protease cleavable peptide linker (06, 07) with or without extra amino acid sequence as a spacer (08, 09, 10 and/or 11) on both the N and C terminals of the protease cleavable linker (06, 07). Domain A and domain B peptides can block the fused antibody or antibody derivative from binding its cognate receptor, ligand or antigen.

For example, domain A peptide and domain B peptide (02, 03) may be interleukin 15 variant and its cognate receptor alpha respectively. Domain A peptide could be a full length of interleukin 15 (Sequences #1, #2) or truncated sequences of interleukin 15 (Sequence #3) with mutations. Domain B peptide can be a full length of interleukin 15 receptor alpha (Sequence #4) or a truncated interleukin receptor alpha (Sequences #5, #6).

Domain A peptide can fuse to either the N terminal of the light chain or the N terminal of the heavy chain of an antibody or one of antibody derivatives, and correspondingly the domain B peptide can fuse to either the N terminal of the heavy chain or the N terminal of the light chain of an antibody or an antibody derivative.

Mask domain A and domain B (02, 03) can be peptide variants of interleukin 15 and peptide variants of interleukin 15 receptor alpha, with certain modifications in their sequences through mutagenesis and truncation, so that they have diminished biological function. The mask domains (02, 03) in the inactive form of masked antibody or masked antibody derivative 04/05/05F are in close proximity to the binding site of an antibody or an antibody derivative 04/05/05F wherein the binding site is specific for the target. In some embodiments, the target comprises a tumor antigen.

Specifically, the heterologous domain A and domain B peptides (02, 03) link to the N terminus of the light and heavy chains of an antibody or an antibody derivative 04/05/05F through a protease cleavable peptide (06, 07) with a spacer or without a spacer (08, 09, 10 and/or 11) can block the antibody variable domains of both light and heavy chains from binding its antigen, receptor or ligand. In some embodiments, the spacer (08, 09, 10 and/or 11) may be a short amino acid sequence and may locate on both ends of the N terminal and C terminal of the protease cleavable peptide (06, 07). The spacer may be any short amino acid sequences, from 2 to 5, or 5 to 10, or 10 to 30, and may comprise (GS)n, (GGS)n, (GGGS)n, (GGGGS)n, (SG)n, (SGG)n, (SGGG)n, (SGGGG)n, (GSGS)n (GSG)n or any other artificial or natural short amino acid sequences.

The protease cleavage peptide linker (06, 07) may be recognized by a serine protease, a cysteine protease, an aspartate protease, a threonine protease, a glutamic acid protease, a metalloproteinase, a gelatinase, an asparagine peptide lyase, or any combinations thereof. The protease cleavable peptide (06, 07) comprises a selected amino acid sequence sensitive to one or multiple proteases. The protease cleavable peptide sequence (06, 07) may be fused between the C terminals of the mask domain or domains (02, 03) and the N terminals of the antibody light chain and heavy chain. The same cleavable peptide sequences (06, 07) may be fused respectively to both the light chain and the heavy chain of antibody or antibody-based therapeutics 04/05/05F, or different protease cleavable peptide sequences (06, 07) may fuse respectively to the light chain and to the heavy chain of antibody or antibody-based therapeutics 04/05/05F.

In one embodiment, the protease cleavable sequence (06, 07) contains two cleavable motifs, either the same or different in a tandem orientation. The protease cleavable peptide sequence (06, 07) fuses to the N terminal of the antibody light chain and the heavy chain respectively. In one embodiment, a protease cleavable sequence (06/07) fuses to the N terminal of an antibody heavy chain, and a protease uncleavable sequence fuses to the N terminal of an antibody light chain. Conversely a protease cleavable sequence (06/07) fuses to the N terminal of antibody light chain, and a protease uncleavable sequence fuses to the N terminal of an antibody heavy chain. The protease cleavable sequence (06, 07) may be selected based on a protease that is co-localized or present in tissue where the activity of the unmasked (activated) antibody or antibody-based therapeutics 04/05/05F is desired or targeted. In some embodiments, the protease cleavage sequence (06, 07) may be recognized by a serine protease, a cysteine protease, an aspartate protease, a threonine protease, a glutamic acid protease, a metalloproteinase, a gelatinase, or an asparagine peptide lyase. In some embodiments, the protease cleavage sequence (06, 07) may be recognized by a Cathepsins, a pepsin, a matriptase, a legumain, a matrix metalloprotease (MMP), a MMP1, a MMP2, a MMP3, a MMP7, a MMP8, a MMP9, a MMP10, a MMP11, a MMP12, a MMP13, a MMP14, an ADAMS, an ADAM10, an ADAM12, an urokinase plasminogen activator (uPA), an enterokinase, a prostate-specific target (PSA, hK3), an interleukin-1β converting enzyme, a thrombin, a FAP (FAP-α), a dipeptidyl peptidase, a type II transmembrane serine protease (TTSP), and a neutrophil elastase.

As described above, embodiments of the invention provide an expression system used for producing the antibody-based molecule or complex as described above. The expression system may include (1) an isolated nucleic acid molecule encoding the antibody-based molecule or complex 01 as described above or components thereof including the first mask peptide, the second mask peptide, the full-length antibody with a specific binding ability or a fragment thereof that maintains said specific binding ability, the first protease cleavable linker, and the second protease cleavable linker; (2) a vector comprising the isolated nucleic acid molecule of (1); and/or (3) a host cell transformed with the vector of (2). One embodiment provides a polynucleotide encoding the conditionally activated binding protein 01 as described above. One embodiment provides a vector comprising the polynucleotide. One embodiment provides a host cell transformed with the vector.

One embodiment provides a process of producing the conditionally activated binding antibody or antibody derivative protein 01 as described above. The process includes culturing a host transformed or transfected with a vector comprising a nucleic acid sequence. One embodiment provides a process of producing the masked antibody or masked antibody-based therapeutics 01, said process comprising culturing a host transformed or transfected with a vector comprising a nucleic acid sequence of the masked antibody or masked antibody-based therapeutics 01.

For example, polynucleotides encoding the mask domain A and domain B (02, 03) may be constructed by methods well known in the prior art, e.g. molecular cloning, such as by combining the genes encoding the various domains within the masked antibody or masked antibody-based therapeutics 04/05/05F. One embodiment provides a host cell transformed with the vector. The masked antibody or the masked antibody-based therapeutics 01, in some embodiments, are produced by introducing a vector encoding the protein 01 into a host cell, including but not limiting to, CHO, CHK1, HEK293, Expi293 or ExpiCHO and culturing said host cell under conditions whereby the protein 01 is expressed.

Exemplary embodiments of the invention provide a pharmaceutical composition comprising (i) the conditionally activated binding antibody or antibody derivative protein 01, the polynucleotide thereof, the vector thereof, or the host cell thereof; and (ii) a pharmaceutically acceptable carrier. In some embodiments, the pharmaceutical composition comprises a therapeutically effective amount of the masked antibody or masked antibody-based therapeutics 01. The carrier does not interfere with the effectiveness of the biological activity of the ingredients and is not harmful to the patient to whom it is administered. Examples of suitable pharmaceutical carriers are well known in the art and include phosphate buffered saline solutions, water, emulsions, various types of wetting agents, sterile solutions.

One embodiment provides a method for the treatment or amelioration of a proliferative disease or a tumorous disease, comprising the administration of conditionally activated binding antibody or antibody derivative protein 01 to a subject (such as human) in need of such a treatment or amelioration. The invention provides a method of inhibiting tumor growth or progression in a subject who has a tumor, comprising administering to the subject an effective amount of the pharmaceutical composition as described herein. The invention provides a method of inhibiting or preventing metastasis of cancer cells in a subject, comprising administering to the subject in need thereof an effective amount of the pharmaceutical composition as described herein. The invention provides a method of inducing tumor regression in a subject in need thereof, comprising administering to the subject an effective amount of the pharmaceutical composition as described herein. In some embodiments, the subject is a human. In some embodiments, the method further comprises administration of an agent in combination with the masked antibody or masked antibody-based therapeutics 01.

Masked antibody or masked antibody-based therapeutics 01 has significantly reduced binding affinity, reduced biological function and reduced cytotoxicity (e.g. antibody drug conjugate). Once the mask domains (02, 03) are removed from the masked antibody, antibody-based therapeutic 01 by protease cleavage, the antibody or the antibody-based therapeutic binding and function will be fully restored. Masked antibody or masked antibody-based therapeutics 01 does not reduce their expression, purification, and pharmacokinetics, nor binding affinity after the unmasking. These features offer the masked antibody or masked antibody-based therapeutics 01 a significant advantage over a naked antibody (unmasked antibody) 04/05/05F in avoiding the potential on-target off-site toxicity in unwanted location.

### EXAMPLES

### Example 1: Preparation and Characterization of Mask Domain A and Domain B (02, 03)

This example provided IL-15 variants (e.g. human IL-15 variants) of domain A that have little or no binding to the IL-15 signaling receptors comprised of IL-2 receptor beta (CD122) and the common gamma chain (CD132), as compared to the wild-type human IL-15 poly peptide or a wild-type IL-15 receptor alpha-IL-15 fusion polypeptide. See Table 1.

The isolated human interleukin 15 (IL-15) variant comprises amino acid substitution at positions a) 8, 65 and 69; or b) 8, 65 and 108; or c) 8, 65, 101 and 108; or d) 8, 65, 101, 108 of SEQ ID NO: 3, and further comprises one or more amino acid substitutions at positions 69, 104 and/or 6 of SEQ ID NO: 3. The IL-15 variant has little or no binding to the human IL-2 receptor beta/gamma (IL-2RBY) as compared to the wild-type human IL-15 polypeptide or a wild-type IL-15 receptor alpha-IL-15 fusion polypeptide.

TABLE 1

| | | |
|---|---|---|
| Amino acid mutation | EC50 | Function |

| | |
|---|---|
| wild | 100 |
| D8S | n/a |
| D8A | |
| D8C | n/a |
| N65A | |
| N65Q | n/a |
| N65K | n/a |
| N65S | |
| L69R | n/a |
| L69A | |
| Q101D | n/a |
| Q101A | n/a |
| Q101S | n/a |
| V104P | n/a |
| V104A | n/a |
| Q108A | |
| Q108D | n/a |
| Q108S | n/a |

Cloning, transfection, protein expression and purification of mask domain A and domain B (02, 03):

Domain A and domain B genes were synthesized by IDT DNA (San Diego, USA) from public database (Sequences #1-#10) using the primer (Table 2) pairs #1/#2, #3/#2 and #4/#2 for amplification of domain A gene fragments, and primer pairs #5/#6 and #5/#7 for amplification of domain B gene fragments. The synthesized domain A and domain B gene fragments were cloned into endonucleases NheI/HindIII (NEB Lab, USA) pre-cut mammalian expression vector pcDNA3.1 with in-fusion cloning kit (Takara, USA) respectively. The right clones with domain A gene and domain B gene were verified by DNA sequencing (MCILab, USA). Mutants of domain A gene, single mutation, double mutation, triple mutation or tetra mutation were generated by site-mutagenesis with exemplary primers (Table 3) and Q5 mutagenesis kit (NEB, USA) and all primers for mutation were designed with NEBaseChanger online server (NEB, USA) and synthesized by IDT DNA (San Diego, USA). The final mutants were verified by DNA sequencing (MCILab, USA).

TABLE 2

| | | Gene |
|---|---|---|
| Primer # | Sequence | fragment |
| #1-Fw | ATGGAGTTTGGGCTGAGCTGGGTTTTCCTCGTTGCTCTTTTTAGAGGTGTCCAGTGT | Seq 2 |
| | GACCCAAGCTGGCTAG ATGCGCATTAGCAAACCGC (SEQ ID NO 11) | |
| #2-Rv | GGTTTAAACTTAAGCTTTTA GCTGGTGTTAATAAACATCTGC (SEQ ID NO 12) | Seq 2 |
| #3-Fw | ATGGAGTTTGGGCTGAGCTGGGTTTTCCTCGTTGCTCTTTTTAGAGGTGTCCAGTGT | Seq 4 |
| | GACCCAAGCTGGCTAG ATGGTGCTGGGCACCATT (SEQ ID NO 13) | |
| #4-Fw | ATGGAGTTTGGGCTGAGCTGGGTTTTCCTCGTTGCTCTTTTTAGAGGTGTCCAGTGT | Seq 6 |
| | GACCCAAGCTGGCTAG AACTGGGTGAACGTGATTAGC (SEQ ID NO 14) | |
| #5-Fw | ATGAAATACCTATTGCCTACGGCAGCCGCTGGATTGTTATTACTCGCGGCCCAGCCGGCCATGG | Seq 8 |
| | CC GACCCAAGCTGGCTAG ATTACCTGCCCGCCG (SEQ ID NO 15) | |
| #6-Rv | GGTTTAAACTTAAGCTTTTA CGCCGCCGAGC (SEQ ID NO 16) | Seq 8 |
| #7-Rv | GGTTTAAACTTAAGCTTTTA GCGAATGCATTTCAGGCTC (SEQ ID NO 17) | Seq 10 |

TABLE 3
Exemplary

| | |
|---|---|
| _D8A:1_FWD | CGTGATTAGCGCGCTGAAAAAAATTGAAG (SEQ ID NO 18) |
| _D8A:1_REV | CTTCAATTTTTTTCAGCGCGCTAATCACG (SEQ ID NO 19) |
| _N65S:1_FWD | TACCGTGGAAAGCCTGATTATTC (SEQ ID NO 20) |
| _N65S:1_REV | GAATAATCAGGCTTTCCACGGTA (SEQ ID NO 21) |
| _Q108A:1_FWD | GCATATTGTGGCGATGTTTATTAACACCAG (SEQ ID NO 22) |
| _Q108A:1_REV | CTGGTGTTAATAAACATCGCCACAATATGC (SEQ ID NO 23) |
| _D8A_N65A:1_FWD | CGTGATTAGCGCGCTGAAAAAAATTGAAG (SEQ ID NO 24) |
| _D8A_N65A:1_REV | GCCAGAATAATCAGCGCTTCCACGGTA (SEQ ID NO 25) |
| _N65A_Q108A:2_FWD | TACCGTGGAAGCGCTGATTATTCTGGC (SEQ ID NO 26) |
| _N65A_Q108A:2_REV | CTGGTGTTAATAAACATCGCCACAATATGC (SEQ ID NO 27) |
| _Q108A_D8A:3_FWD | GCATATTGTGGCGATGTTTATTAACACCAG (SEQ ID NO 28) |
| _Q108A_D8A:3_REV | CTTCAATTTTTTTCAGCGCGCTAATCACG (SEQ ID NO 29) |
| _D8S_N65S:1_FWD | CGTGATTAGCAGCCTGAAAAAAATTGAAGATCTG (SEQ ID NO 30) |
| _D8S_N65S:1_REV | GAATAATCAGGCTTTCCACGGTA (SEQ ID NO 31) |
| _N65S_Q108S:2_FWD | TACCGTGGAAAGCCTGATTATTC (SEQ ID NO 32) |
| _N65S_Q108S:2_REV | GTTTTAGCTGGTGTTAATAAACATGCTCACAATATGC (SEQ ID NO 33) |
| _Q108S_D8S:3_FWD | GCATATTGTGAGCATGTTTATTAACACCAGCTAAAAC (SEQ ID NO 34) |
| _Q108S_D8S:3_REV | CAGATCTTCAATTTTTTTCAGGCTGCTAATCACG (SEQ ID NO 35) |
| _D8S_L69S:1_FWD | CGTGATTAGCAGCCTGAAAAAAATTGAAGATCTG (SEQ ID NO 36) |
| _D8S_L69S:1_REV | GGCTGTTGTTCGCGCTAATAATCAGG (SEQ ID NO 37) |
| _L69S_Q101A:2_FWD | CCTGATTATTAGCGCGAACAACAGCC (SEQ ID NO 38) |
| _L69S_Q101A:2_REV | CAATATGCACAAAGCTCGCCAGAAATTCT (SEQ ID NO 39) |
| _Q101A_Q108S:3_FWD | AGAATTTCTGGCGAGCTTTGTGCATATTG (SEQ ID NO 40) |
| _Q101A_Q108S:3_REV | GTTTTAGCTGGTGTTAATAAACATGCTCACAATATGC (SEQ ID NO 41) |
| _Q108S_D8S:4_FWD | GCATATTGTGAGCATGTTTATTAACACCAGCTAAAAC (SEQ ID NO 42) |

Domain A/domain B heterodimer proteins, and domain A mutant/domain B heterodimer proteins were produced from HEK293 FreeStyle™ Expression Medium (Invitrogen, USA) after co-transfection of synthetic domain A and domain B, or mutant A and domain B vectors respectively.

The culture supernatants were applied to protein A Sepharose columns (Cytiva, USA) after filtration with 2 um unit (Millipore, USA) 5 days post transfection. The column was washed with PBS, and protein was then eluted with eluting buffer (0.1 M sodium citrate buffer, pH 3.0). Collected fractions were neutralized with 1 M Tris pH 9.0. For protein polishing, size-exclusion chromatography (HiLoad® 16/600 Superdex® 200 pg, cytiva, USA) was performed with AKTA explorer (GE HEALTH, USA) after cleaning the column and equilibrated with PBS. The protein A purified protein was concentrated to −8 mg/ml and filtered with 0.2 uM syringe filter (Millipore, USA) and injected into the prepared column with flow rate at 1 ml/min. Protein flow-through was collected using Fac95 fraction collector. Finally, purified samples were polled together, and the protein concentration was measured at UV280 nm (Nanodrop, ThermoScientific, USA). Purity of the eluted antibody fraction was analyzed by sodium dodecyl sulfate polyacrylamide gel electrophoresis (SDS-PAGE) on 12% gradient gels under reducing or non-reducing conditions. Bands were visualized by Coomassie brilliant blue staining. The homogeneity was analyzed with analytic SEC-HPLC with TSKgel G3000SWXL size exclusion HPLC column (TOSOH, USA) in Agilent 1100 system (Agilent, USA) running with phosphate buffer pH6.8 plus 150 mM NaCl at Iml/min flow rate.

Function evaluation of modified domain A and domain B complex using human PBMC: It is known that IL-15 promotes CD8+ memory T, natural killer (NK), and NKT cells proliferation, survival, and homeostasis. IL-15 causes T cell activation indicated by upregulation of the membrane surface expression of CD69 and cytokine release including IFNγ. CD69 is an early activation marker of T cells. As measured by the percentage of CD69 surface expression to reflect the percentage of CD8+ T cell activation.

To compare the activity of the IL-15 wild to IL-15 variants in activating CD8+ T cell, this assay was performed essentially as follows: human PBMC (Stem Cell Technologies, USA. Catalog number 70500) were plated with a cell number of 2×10\hat{ }5/well in 96 well round-bottom cell culture plate in RPMI-1640 medium supplemented with 10% FBS, 1% penicillin, and streptomycin. The IL-15 variants or IL-15 wild were 3-times titrated with medium and 5 μL were added to the wells. Each concentration was repeated in triplicate, blank wells (added with only medium) were used as a blank control. Cell plates were cultured in the incubator for 3 days. Then the cells were centrifuged, and the cell pellet was stained with anti-CD3 Ab, anti-CD8 Ab, and anti-CD69 Ab (BioLegend, USA) in FACS buffer for 30 minutes. They were washed twice with FACS buffer and acquired by flow cytometry with Attune (Thermo-Fisher Scientific). Data were analyzed with FlowJo software. The percentage of CD69+ cells in CD8+ T cells was plotted with Prism 8 (Graphpad, USA).

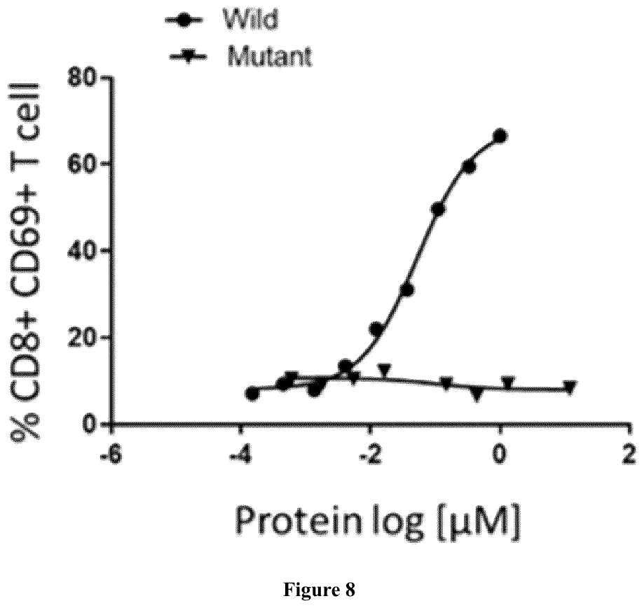

FIG. 8 shows the function of IL-15 mutant and IL-15 wild using human PBMC assay. As shown in FIG. 8, the domain A (IL-15) variant (mutant) had no detectable activity to stimulate T cell activation as compared to the IL-15 wild. The result demonstrated that the domain A (IL-15) mutation directly abolished IL-15 from activating CD8+ T cells.

### Example 2: Preparation and Characterization of Masked Antibody

To construct anti-PD-L1 antibody and masked-a-PD-L1 antibody: anti-PD-L1 antibody heavy chain and light chain variable fragments were synthesized using published a-PD-L1 mAb Atezolizumab (TABLE 5) gene sequences by gene synthesis (GENSCRIPT, USA). Amplification of human IgG1 constant region fragment and of human kappa constant region fragment was done with synthesized primer pairs #8/#9 and #10/#11 (IDT DNA, USA) using hIgG1 and human kappa plasmids as templates respectively. The final constructs were accomplished by in-fusion cloning of the anti-PD-L1 heavy variable gene, hIgG1 constant PCR fragment and endonuclease NheI/HindIII digested pcDNA3.1 plasmids. The final clones were verified by DNA sequencing (MCILab, USA).

For masked-a-PD-L1 antibody construction, the mask domain gene fragments were amplified by PCR with primers containing protease substrate sequences from templates of constructed pcDNA3.1 expression vectors with primers #1/#2, #2/#3, #2/#4 for domain A and #5/#6 and #5/#7 for domain B (see Table4). The full length of a-PD-L1 antibody heavy chain and light chain were amplified with primer pairs #12/#13 and #13/#14 (see Table 4). In-fusion cloning of mask domain A+protease substrate linker fragment to the N terminal of heavy chain of a-PD-L1 antibody PCR fragment into NheI/HindIII pre-cut vector pcDNA3.1, and of mask domain B+protease substrate linker fragment from PCR to the N terminal of light chain of a-PD-L1 antibody PCR fragment into the NheI/HindIII pre-cut vector pcDNA3.1. The right clones were screened and selected after mini plasmid preparation and DNA sequencing (MCI Lab, USA).

The proteins were produced from HEK293 FreeStyle™ Expression Medium (Invitrogen, USA) after co-transfection of mask-a-PD-L1 heavy chain and light chain plasmids to HEK293 FreeStyle cells. The culture supernatants were applied to protein A Sepharose columns (Cytiva, USA) 5 days post transfection. The column was washed with PBS, and protein was then eluted with eluting buffer (0.1 M sodium citrate buffer, pH 3.0). Collected fractions were neutralized with 1 M Tris pH 9.0. The eluted protein was concentrated to −8 mg/ml with Concentrator (Pall, USA) and filtered with 0.2 uM syringe filter and injected into the prepared column with flow rate at iml/min. Protein flow through was collected using Fac95 fraction collector. Finally, purified samples were pooled together, and the protein concentration was measured at UV280 nm (Nanodrop, ThermoScientific, USA). Protein was further polished with size-exclusion chromatograph with column (HiLoad® 16/600 Superdex® 200 pg, cytiva) using AKTAexplorer (GE HEALTH, USA) after cleaning and equilibrated with PBS. Concentrated protein was filtered and injected into prepared size-exclusion column at 1 ml/min flow rate with PBS, and flow-through fraction was collected. Finally, collected fractions were polled together and the protein concentration was determined at UV280 nm with Nano-drop (Thermo Scientific, USA). Purity of the antibody was analyzed by sodium dodecyl sulfate polyacrylamide gel electrophoresis (SDS-PAGE) on 1200 gradient gels under reducing or non-reducing conditions. Bands were visualized by Coomassie brilliant blue staining. The homogeneity was analyzed with analytic SEC with TSKgel G3000SWXL size exclusion TIPLC column (TOSOH, USA) in Agilent 1100 system (Agilent, USA).

TABLE 4

| Primer# | Sequence |
|---|---|
| #1-Fw | ATGGAGTTTGGGCTGAGCTGGGTTTTCCTCGTTGCTCTTTTTAGAGGTGTCCAGTGTG |
| | ACCCAAGCTGGCTAGATGCGCATTAGCAAACCGC (SEQ ID NO 43) |
| #2-Rv | GTGGTTGGCGCTTCTGCCGCTCAGGCT GCTGGTGTTAATAAACATCTGC (SEQ ID NO |
| | 44) |
| #3-Fw | ATGGAGTTTGGGCTGAGCTGGGTTTTCCTCGTTGCTCTTTTTAGAGGTGTCCAGTGT |
| | GACCCAAGCTGGCTAG ATGGTGCTGGGCACCATT (SEQ ID NO 45) |
| #4-Fw | ATGGAGTTTGGGCTGAGCTGGGTTTTCCTCGTTGCTCTTTTTAGAGGTGTCCAGTGT |
| | GACCCAAGCTGGCTAG AACTGGGTGAACGTGATTAGC (SEQ ID NO 46) |
| #5-Fw | ATGAAATACCTATTGCCTACGGCAGCCGCTGGATTGTTATTACTCGCGGCCCAGCCGG |
| | CCATGGCCGACCCAAGCTGGCTAG ATTACCTGCCCGCCG (SEQ ID NO 47) |
| #6-Rv | GTCGCTTCTGCCGCTCAGGCCCAGAGGGCTGCCCGCCGCCGAGC (SEQ ID NO 48) |
| #7-Rv | GTCGCTTCTGCCGCTCAGGCCCAGAGGGCTGCCGCGAATGCATTTCAGGCTC (SEQ ID |
| | NO 49) |
| #8-Fw | GCTAGCACCAAGGGCC (SEQ ID NO 50) |
| #9-Rv | GGTTTAAACTTAAGCTTTTATTTACCCGGAGACAGG (SEQ ID NO 51) |
| #10-Fw | CGTACGGTGGCTGCAC (SEQ ID NO 52) |
| #11-Rv | GGTTTAAACTTAAGCTTCTAACACTCTCCCCTGTTGAAG (SEQ ID NO 53) |
| #12 | CAGAAGCGCCAACCAC (SEQ ID NO 54) |
| #13 | AGGCTGATCAGCGGTTTAAAC (SEQ ID NO 55) |
| #14 | GAGCGGCAGAAGCGAC (SEQ ID NO 56) |

TABLE 5

| Anti-PD-L1 | Sequence |
|---|---|
| Heavy V Chain | EVQLVESGGGLVQPGGSLRLSCAASGFTFSDSWIHWVRQAPGKGLEWVAWISPYGGSTYYADSVKGRF |
| | TISADTSKNTAYLQMNSLRAEDTAVYYCARRHWPGGFDYWGQGTLVTVSS (SEQ ID NO 57) |
| CAGAAGCGCC | GAG GTT CAG CTC GTT GAG TCC GGC GGA GGC CTC GTA CAG CCA GGG GGG TCT TTG CGA |
| AACCAC (SEQ | CTG TCC TGC GCG GCA TCT GGG TTC ACC TTC TCC GAT AGC TGG ATA CAT TGG GTC AGG |
| ID NO 58) | CAA GCT CCT GGA AAA GGG CTG GAG TGG GTT GCG TGG ATT TCC CCT TAT GGC GGG TCT |
| | ACT TAT TAT GCG GAC AGC GTG AAG GGC AGA TTC ACC ATT TCC GCT GAT ACT TCA AAG |
| | AAT ACA GCT TAC TTG CAG ATG AAC TCA CTC CGC GCG GAA GAT ACT GCA GTC TAC TAC |
| | TGC GCT CGG AGA CAT TGG CCA GGG GGG TTC GAC TAT TGG GGC CAG GGC ACC CTG GTA |
| | ACC GTC AGT TCA (SEQ ID NO 59) |
| | GCTAGCACCAAGGGCC (SEQ ID NO 60) |
| Light V Chain | DIQMTQSPSSLSASVGDRVTITCRASQDVSTAVAWYQQKPGKAPKLLIYSASFLYSGVPSRFSGSGSGTD |
| | FTLTISSLQPEDFATYYCQQYLYHPATFGQGTKVEIK (SEQ ID NO 61) |
| GAGCGGCAGA | GAT ATA CAG ATG ACG CAA TCC CCA TCC TCT TTG TCC GCG TCC GTT GGT GAT CGG GTG |
| AGCGAC (SEQ | ACC ATA ACC TGC CGA GCA AGC CAA GAC GTT AGC ACG GCT GTA GCA TGG TAC CAG CAA |
| ID NO 62) | AAA CCA GGC AAA GCA CCA AAA CTT CTC ATA TAC TCT GCA AGT TTT CTC TAT AGT GGA |
| | GTG CCT AGT AGG TTC AGT GGC TCA GGC TCT GGA ACA GAC TTT ACC CTT ACT ATC AGC |
| | TCA CTT CAG CCA GAA GAT TTC GCC ACA TAT TAC TGC CAG CAG TAC CTT TAC CAT CCT |
| | GCG ACT TTC GGT CAG GGG ACC AAA GTA GAA ATT AAG (SEQ ID NO 63) |
| | CGTACGGTGGCTGCAC (SEQ ID NO 64) |

### Determination of Antibody Affinity to its Ligand

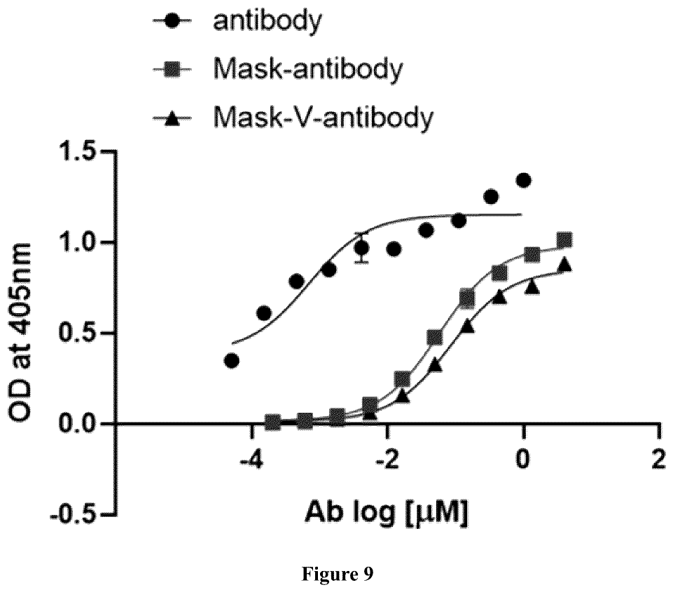

The binding affinity of masked antibody was assessed by ELISA in comparison with non-masked antibody. The ELISA plate was coated with 50 ul each well of recombinant human PD-L1 (Acrobio, USA) at 1 g/ml at 4° C. overnight. After blocking with 1% BSA in PBS 1 hr at room temperature, the plate was washed 3 times with PBS-T (Tween 20 at 0.050%). A serial diluted masked antibody or non-masked antibody was loaded with 100 ul each well to the plate and incubated for 2 hours at room temperature. The plate was washed 3 times with PBS-T. Then Human IgG1 antibody is used as a negative control. After plate washing, an anti-human IgG-Fc-AP conjugated antibody at 1:2000 dilution was added to the individual well of the plate and incubated for 45 min at room temperature. After plate wash 3 times with PBS-T, the AP substrate pNPP (Thermo, USA) was added 100 ul each well and the optical densities were obtained at 405 nm wavelength with a spectrophotometer (BioTec, USA). The data were analyzed and plotted with Prism 8 (GraphPad, USA). FIG. 9 shows in vitro binding assay comparing the masked antibody to the un-masked counterpart antibody. As shown in FIG. 9, the masked antibody has dramatically lower affinity to human PD-L1 with comparison to the non-masked one did. The result suggested that the mask domain effectively hindered the antibody binding function.

In vitro function assay using reporter cell lines:

Jurkat-PD1 T cells and Raji-PD-L1 B cells were maintained in RPMI medium (RPMI 1640 supplemented with 10% fetal bovine serum, 100 U/mL of Penicillin, and 100 mg/mL of Streptomycin) at 37° C./5% CO2.

Cell preparation: Prepare Jurkat-PD1(2×10\hat{ }6 cell/ml) and Raji-PD-L1 (1×10\hat{ }6 cell/ml) cells; Load 50 ul each of the cells/per well to 96-well cell culture plate (Corning, USA); Load 50 ul of pre-serial diluted mask antibody and non-mask antibody into the well with pre-loaded cells, and leave the plate in the incubator for 20-30 min. Load 50 ul of aCD3/aCD19 (4 ng/ml) into each well of the plate. Leave the plate in the incubator at 37° C./5% CO2 for 2 days. The cell culture plate was centrifuged at 1000×g for 2 min. The cultured supernatants were transferred into a fresh 96 well plate for later use.

IL-2 DETECTION ELISA: Bio-legend ELISA MAX™ Deluxe Set for IL-2 was used for this assay by following the manufacturer's instruction. In brief, a 96-well ELISA plate (Greiner, USA) was coated with 50 ul each of 200× PBS-diluted anti-IL-2 antibody and sat at room temperature for 2 hours with shaking. Then the ELISA plate was washed 3 times with PBS-T (Tween-20 at 0.05% vol/vol) and added 50 ul of blocking buffer (3% BSA in PBS pH7.4) at room temperature for 1 hour. Then the plate was washed 3 times with PBS-T. The cell culture supernatant was loaded 100 ul each well after a serial dilution with ELISA blocking buffer and incubated at room temperature for one and half hours. Then the plate was wished 3 times with PBS-T. The detection antibody diluted with blocking buffer was added to each well and the plate was incubated for 1 hour at room temperature. After 3 times of washing, the plate was loaded with 100 ul each well of avidin-TRP and incubated for 45 min at room temperature. The plate was washed 3 times and then 100 ul of substrate was added to each well and read at 405 nm with a spectrophotometer (BioTec, USA). The data were analyzed and plotted with Prism 8 (GraphPad, USA).

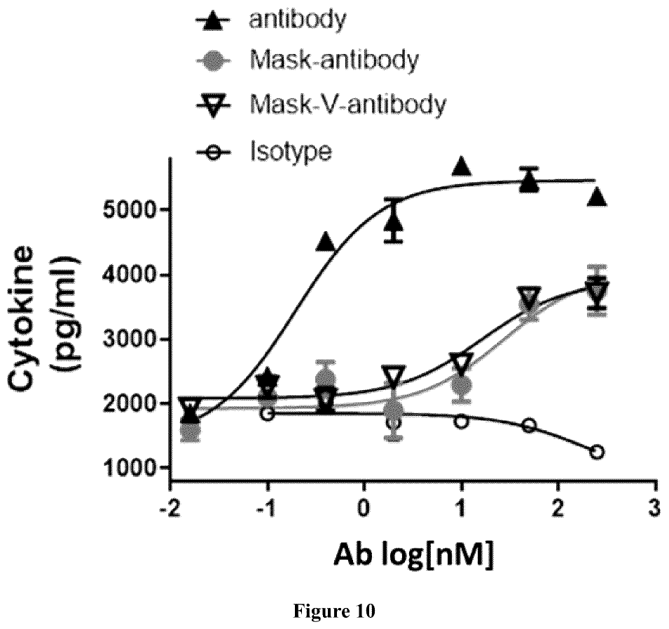

FIG. 10 shows a cell-based function assay comparing the masked antibody to the un-masked antibody. As shown in FIG. 10, the masked antibody has dramatically reduced IL-2 production with comparison to the non-masked one has done. The result suggested that the mask domain effectively hindered the antibody function from stimulating IL-2 production.

### Example 3: Cleavage of Protease Substrate (06, 07) by Different Proteases

Metalloproteases and other proteases are upregulated in many cancers. The MMP2 (AcroBiosystems, USA) and MMP9 (R&D System, USA) were activated via incubation with 4-aminophenyl mercuric acetate (APMA) at 37C for 1 hour up to 24 hours (overnight) by following the manufacturer's instruction. Active MMP14 and uPA were from R&D systems and from AcroBiosystems. To assess the cleavage profile of the protease substrate sequences used between the mask domain and the antibody, masked antibodies (5 g) were incubated at 37° C. overnight with or without 400 pmol/min normalized proteases as indicated by manufacturer's instruction. Cleavage bands were separated by sodium dodecyl sulfate polyacrylamide gel electrophoresis (SDS-PAGE) on 12% gradient gels (SnapGen, USA) under non-reducing conditions. Bands were visualized by Coomassie brilliant blue staining.

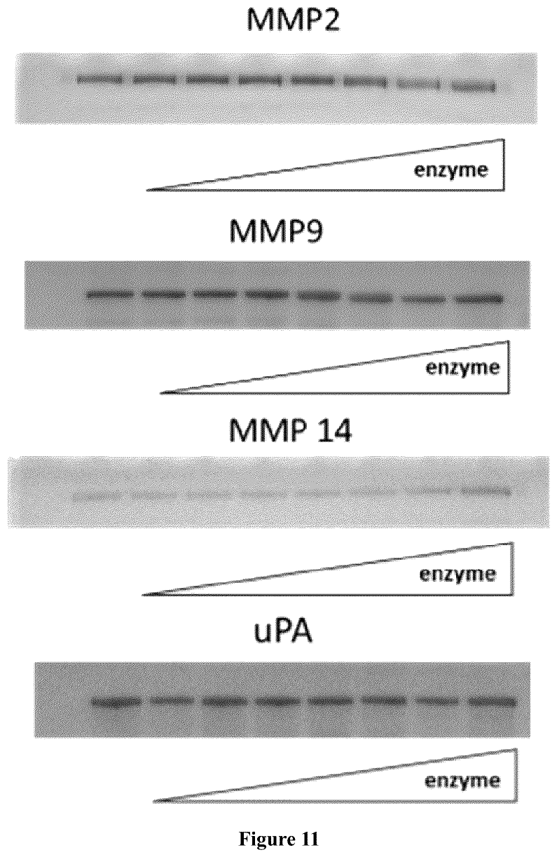

The data together in FIG. 11 demonstrated that MMP2 and MMP9 protease substrate sites in the linker (06, 07) are cleavable and showed dose-dependent manner. MMPP14 and uPA proteases did not cleave the substrate sequence used in this masked antibody molecule.

### Example 4: Masked Antibody CD47

### Cloning, Transfection and Protein Generation

The variable region of a-CD-47 antibody heavy chain and light chain will be synthesized (IDT DNA, San Diego, USA) according to their nucleic acid sequences (shown below) and fused with pre-cut human IgG1 and human kappa constant region in pcDNA3.1 expression vectors respectively (Takara, USA) and the final clones will be verified by DNA sequencing (MCI Lab., USA). The antibody proteins will be produced from HEK293 FreeStyle™ Expression Medium (Invitrogen) after transfection of synthetic heavy and light chain vectors Soluble CD-47 will be produced by CHO-K1 cells transfected with cDNA encoding the extracellular region of CD-47.

The culture supernatant will be applied to protein A Sepharose columns (GE Healthcare). The column will be washed with PBS, and protein is then eluted with eluting buffer (0.1 M sodium citrate buffer, pH 3.0). Collected fractions are neutralized with 1 M Tris pH 9.0. Finally, purified samples are dialyzed against PBS. Purity of the eluted antibody fraction is analyzed by sodium dodecyl sulfate polyacrylamide gel electrophoresis (SDS-PAGE) on 12% gradient gels under reducing or non-reducing conditions. Bands are visualized by Coomassie brilliant blue staining.

The stability of masked anti-CD47 antibodies will be assessed in vivo using intravenous administration to BALB/c mice. Antibodies are dosed at 5 mg/kg. At the given time point (3 days), plasma is collected from dosed mice. Human antibody is purified from plasma using MabSelect resin. Captured antibody is reduced and separated by SDS-PAGE, then probed by Western blot using an HRP-conjugated anti-human Fc antibody. The percent cleaved antibody is assessed by densitometry of bands corresponding to masked and non-masked light and heavy chains, which differ in size. The stability of masked anti-CD47 antibodies is also assessed in vitro by incubation of human plasma with masked anti-CD47 antibodies and non-masked anti-CD-47 antibodies. After certain days of incubation at 37c, run western blot to detect the masked and unmasked light and heavy chains.

To test the ability of masked anti-CD47 antibody having improved pharmacokinetics and tolerability than non-masked anti-CD47 antibodies, a series of IV single dose studies will be conducted in cynomolgus macaques. The animals are monitored daily for toxicity symptoms and blood will be drawn at certain time points, 0, 30 min, 60 min, 4 hrs, 8 hrs, 16 hrs, 24 hrs, 48 hr, 72 hrs for testing masked anti-CD-47 antibody concentration in blood and get its circulation half time compared to non-masked-a-CD-47 antibody by ELISA. In brief, a 96-well ELISA plate (Greiner, USA) will be coated with anti-Domain B antibody (anti-IL15Ralpha, R&D Systems, USA) at 2 ug/ml overnight at 4c. The plate will be blotted with blocking buffer (3% BSA in PBS) at room temperature for 1 hr. The plate will be washed 3 times with PBS-T. The plate will be added with the serial diluted sera from the studies animals and incubated at room temperature for one and half hrs. The plate will be washed 3 times with PBS-T. The plate will be added with mouse anti human IgG-Fc-HRP 2ndary antibody at the concentration recommended by the manufacturer for 1 hr. The plate will be washed 3 times, and the substrate will be added to the plate and read at 405 nm with a spectrophotometer (BioTec, USA). The data will be analyzed and plotted with Prism 8 (GraphPad, USA). The data will demonstrate that the masked a-CD-47 antibody has longer circulation half time than non-masked a-CD-47 antibody.

Evaluation of protease activity by in situ gel zymography of a panel of cynomolgus macaque and human tissues will indicate protease activity levels across species. Therefore, the results of this study will guide if cynomolgus macaque will represent a relevant species for evaluating the impact of masking on Anti-CD47 antibody pharmacokinetics and tolerability.

Hematotoxicity is one of main side effects caused by anti-CD-47/SIRPa pathway. Healthy C57-hCD47/hSIRPa mice (male and female, about 12-week-old) are injected i.p. with a single dose of anti-CD47 antibodies (20 mg/kg, mIgG2a isotype) or PBS. Blood is drawn from the retro-orbital plexus and collected in di-potassium-EDTA anticoagulation tubes at 3 hr after injection. The hematological analyses are performed using the ADVIA 2120 Hematology System (Siemens) to assess the complete blood count. This analysis is carried out at CRO. It is expected that the masked anti-CD-47 antibody has little, or no significant reduction of blood cell account compared to the non-masked anti-CD-47 antibody.

Antibody dependent cellular phagocytosis (ADCP) will be conducted using Bone marrow-derived macrophages (BMDMs) from C57-hCD47/hSIRPa mice are used as effector cells in this assay. To prepare BMDMs, mouse bone marrow cells are collected from the tibia and femurs of C57-hCD47/hSIRPa mice, the cells are subsequently stimulated by adding L929-cell-culture-supernatants (containing granulocyte macrophage-colony-stimulating factor (GMCSF) that secreted by L929 cells) to the medium and cultured on a 24-well tissue culture plate for 7 days. Tumor cells are labeled with carboxy fluorescein succinimide ester (CFSE) following the manufacturer's instructions (Thermo Scientifc) and used as target cells. The BMDMs are labeled with anti-mouse F4/80-Alex Fluor647 (Thermo Scientifc) prior to incubation with tumor cells. The CFSE-labeled tumor cells will be incubated with different antibodies at room temperature for 15 min and then added to the labeled BMDMs using an effector-to-target ratio of about 1:1. Cells are incubated at 37° C. for 2 h in RPMI1640 medium supplemented with 10% heat-inactivated FBS. During the phagocytosis assay, the pH of the medium is adjusted to pH 7.4 and 6.8 using HEPES and PIPES, respectively. The phagocytosis of tumor cells by macrophages is measured via confocal microscopy.

Anti-tumor activity will be estimated using human tumor xenograft models, 6-8 weeks old female NOD-SCID mice will be inoculated subcutaneously (s.c.) with 1×10\hat{ }6 Raji cells on the right lower flank. When tumors reached about 50 mm3, mice are intraperitoneally (i.p.) injected with anti-CD47 antibodies or masked anti-CD47 antibodies (10 mg/kg) or PBS as control started on day 8 after inoculation, 2 doses per week for 3 weeks. For syngeneic mouse models, 6-8-week-old male and/or female C57-hCD47/hSIRPa or BALB/c-hCD47/hSIRPa mice are inoculated subcutaneously (s.c.) with 5×, 2×10\hat{ }6 LL/2-hCD47, 2×10\hat{ }5 B16-hCD47, or 3×10\hat{ }5 CT26-hCD47 cells on the right lower flank. After 4 days or once the tumor volume reaches 50 mm\hat{ }3, antibodies, including a-CD-47 antibody, masked-a-CD-47 antibody, isotype control are applied with 1 mg/kg, 3 mg/kg and 10 mg/kg through peritoneal injection, twice a week. The tumor growth will be evaluated twice weekly by direct caliper measurements. Total doses will be 6 times. During the course, the animals will be monitored with clinical index, body weight, physical conditions, food-intake and so on. After 28 days of post inoculation of tumor cells, the animal will be euthanized, and the tumor mass will be weighed.

It is expected that the masked-a-CD-47 antibody will show similar efficacy as the non-masked-a-CD-47 antibody in tumor growth inhibition. The isotype control would not show any tumor growth inhibition role. It would be expected that non-masked-a-CD-47 antibody might induce obvious toxicity even mortality in treated animals compared to masked-a-CD-47 antibody.

Heavy chain Fv:
(SEQ ID NO 65)
QVQLVQSGAEVKKPGASVKVSCKASGYTFTNYNMHWVRQAPGQRLEWMG
TIYPGNDDTSYNQKFKDRVTITADTSASTAYMELSSLRSEDTAVYYCAR
GGYRAMDYWGQGTLVTVSS
Light chain Fv:
(SEQ ID NO 66)
DIVMTQSPLSLPVTPGEPASISCRSSQSIVYSNGNTYLGWYLQKPGQSP
QLLIYKVSNRFSGVPDRFSGSGSGTDFTLKISRVEAEDVGVYYCFQGSH
VPYTFGQGTKLEIK (US11014985B2)

### Example 5: Masked Antibody Ep-CAM-Drug Conjugate

The variable region of a-Ep-CAM antibody heavy chain and light chain will be synthesized (IDT DNA, San Diego, USA) according to their sequences (shown below) and fused with pre-cut human IgG1-constant region and human kappa constant region in pcDNA3.1 expression vectors respectively (Takara Bio, USA) and the final clones will be verified by DNA sequencing (MCI Lab., USA). The antibody proteins will be produced from HEK293 FreeStyle™ Expression Medium (Invitrogen) after transfection of synthetic heavy and light chain vectors Soluble Ep-CAM will be produced by CHO-K1 cells transfected with cDNA encoding the extracellular region of Ep-CAM.

Conjugation of compound with anti-Ep-CAM antibody. Anti-Ep-CAM antibody interchain disulfides will be reduced with an excess of TCEP (20 eq, pH 8, 37° C., 90 minutes), which is removed via buffer exchange with Amicon spin filters (EMD Millipore UFC503096). The disulfides are re-oxidized (with the exception of engineered thiols) using dehydro ascorbic acid (Sigma Aldrich 261556) (20 eq, pH 7.4, RT, 45 minutes, 2 additions). Excess dehydro ascorbic acid is removed via three rounds of dilution and concentration in Amicon spin filters. Propylene glycol is added to the reaction mixture to a final concentration of 50%. A solution of PBD-linker (3 eq) is diluted with propylene glycol and added to the re-oxidized antibody (leq) containing two free thiols at position 239. The reaction is allowed to proceed for 30 minutes then treated with a slurry of activated charcoal in water for 30 minutes. The activated charcoal is removed via filtration and the reaction is further purified using a Nap5 column (GE Healthcare Life Sciences, 17-0853-02). For mixed 4-load mc-MMAE ADCs, antibody interchain disulfides are partially reduced using 2.1 equivalents of TCEP at pH 8 for 60 min. Excess TCEP is then removed by two rounds of dilution and concentration. At that time, 4.4 molar equivalents of drug-linker is added to the partially reduced antibody. Following conjugation excess drug-linker is removed using quadrasil MP thiol resin for 15 min, followed by desalting into PBS, pH 7.4 using a PD-10 gel filtration column. For all ADCs, drug-loading is determined by PLRP-MS and the extent of aggregation is determined by size exclusion chromatography.

Light chain variable region:
(SEQ ID NO 67)
DIVMTQSALSNPVTLGESGSISCRSSKSLLHSNGITYLYWYLQKPGQSP
QLLIYQMSNRASGVPDRFSSSGSGTDFTLKISRVEAEDVGVYYCAQNLE
LPRTFGQGTKLEMK
Heavy chain variable region:
(SEQ ID NO 68)
QIQLVQSGPEVKKPGESVKISCKASGYTFTKYGMNWVKQAPGQGLKWMG
WINTYTEEPTYGDDFKGRFTFTLDTSTSTAYLEISSLRSEDTATYFCAR
FGSAVDYWGQGTLVTVSS

Binding of anti-Ep-CAM antibody with Flow cytometric analysis.

SW480 cells (ATCC, USA) will be grown in complete medium with 10% FBS to confluency in cell culture flask (Corning, USA). Cultured cells are dissociated using Accutase (Innovative Cell Technologies, Inc., San Diego, CA, USA) and aliquoted to a 96-well plate with 5×10\hat{ }3 per well and incubated with either a-Ep-CAM antibody, masked-a-Ep-CAM antibody or Human IgG1 starting 10 g/ml with serial dilution at 4° C. for 1 hr. Then, plate is washed with cold PBS twice, followed by incubation with 10 μg/ml PE-labeled anti-human Fc gamma specific antibody (eBiosciences, San Diego, CA, USA) at 4° C. for 1 hr. then the plate is washed twice with cold PBS. Binding is measured using an Attune (ThermoScientific, CA, USA). Cells incubated with secondary antibody but without primary a-Ep-CAM Ab are used as negative control. The data will be analyzed with Flowjo and plotted with Prism 8 (GraphPad, USA). It is expected that the a-Ep-CAM antibody has a better binding efficiency than the masked-a-Ep-CAM antibody.

Cell viability assays will be performed using Realtime-Glo (Promega). HCT116 cells (5×10\hat{ }3 cells/well) are seeded onto a 96-well culture plate with a clear bottom in culture medium according to vendor protocol. Dilutions of masked or non-masked anti-Ep-CAM ADC PBD-conjugated ADC, ADC, are added to each well (1000 ng/mL to 0 ng/mL) and the samples are incubated for 60 hours at 37° C. Luminescence is measured using a plate reader (Envision 4605). The data is fit using Prism 8 (GraphPad, USA) and an IC50 is calculated. The IC50 values are used to compare changes in cytotoxicity of non-masked anti-Ep-CAM ADC against masked anti-Ep-CAM ADC. The cytotoxicity of masked anti-Ep-CAM ADC antibodies is expected to be significantly reduced as they bind to HCT116 or LoVo cells. Cytotoxicity of masked anti-Ep-CAM ADC antibody is restored to the similar toxicity after removing the mask in vitro as the non-masked anti-Ep-CAM ADC antibody.

Antitumor activity of both a-Ep-CAM antibody and masked-a-Ep-CAM antibody will be evaluated on in vivo HCT116 cell (ATCC, USA) inoculated NOG mouse model. The HCT cell is cultured in growth medium (Gibco #16600) plus 10% FBS and 1% PS. When they are confluence, dissociated using Accutase (Innovative Cell Technologies, Inc., San Diego, CA, USA) and wash once with fresh medium and suspended in 1×10\hat{ }6 cell/ml with PBS. Inoculation of 0.1 ml of HCT116 cells per mouse to the fat pad of NOG mouse. Once the tumor grows to 50 mm\hat{ }3, mice are dosed twice a week through intraperitoneal or intraventricular at dose 0.5 mg/kg, 1 mg/kg, 3 mg/kg, 9 mg/kg. Tumor size is measured twice a week and the tumor volume is calculated based on the equation “V=0.5×L×W\hat{ }2” and the tumor growth curve is graphed over time. It is expected that the masked-a-Ep-CAM ADC has similar effect on the tumor inhibition to the non-masked-a-Ep-CAM ADC after removing the mask in tumor. The tumor inhibition shows a dose-dependent response trend with no or little toxicity compared to non-masked anti-Ep-CAM ADC.

Light chain variable sequence:
(SEQ ID NO 69)
DIVMTQSALSNPVTLGESGSISCRSSKSLLHSNGITYLYWYLQKPGQSP
QLLIYQMSNRASGVPDRFSSSGSGTDFTLKISRVEAEDVGVYYCAQNLE
LPRTFGQGTKLEMK
Heavy chain variable sequence:
(SEQ ID NO 70)
QIQLVQSGPEVKKPGESVKISCKASGYTFTKYGMNWVKQAPGQGLKWMG
WINTYTEEPTYGDDFKGRFTFTLDTSTSTAYLEISSLRSEDTATYFCAR
FGSAVDYWGQGTLVTVSS

### Example 6: Masked a-CD-47/a-Ep-CAM Bi-specific Antibody

Cloning: To construct scFv-Fc of a-Ep-CAM antibody and masked-scFv-Fc of a-Ep-CAM antibody, VH and VL or masked VH and VL fragments will be amplified by PCR from light chain and heavy chain of a-Ep-CAM plasmids, or by PCR from light chain and heavy chain of masked-a-Ep-CAM plasmids. The “hinge-Fc” fragment will be generated by PCR using the heavy chain of a-Ep-CAM plasmid. Infusion cloning will be employed to the construction of scFv-Fc and masked-scFv-Fc of a-Ep-CAM antibody with pre-cut mammalian expression vector pcDNA3.1. The anti-CD-47/anti-Ep-CAM bi-specific antibody will be built with “knob-into-hole” technology (8). To make the “hole” arm of a-CD47 antibody and masked a-CD-47 antibody, 3 site mutagenesis will be introduced in both a-CD-47 antibody's heavy chains to make S366, A368 and V407 respectively. To make the “knob” arm of a-Ep-CAM scFv-Fc and masked a-Ep-CAM scFv-Fc antibodies, site mutagenesis of amino acid will be introduced to amino acid W366. All mutagenesis will be accomplished with NEBase Changer online software for primer design and with Q5 site-directed mutagenesis kit for the reaction based on the manufacturer's instruction (NEB Bio lab). All clones will be expanded, and their plasmid DNA will be extracted for DNA sequencing validation (MCI Lab, USA). The antibody proteins will be produced from HEK293 FreeStyle™ Expression Medium (Invitrogen) after transfection of constructed heavy and light chain vectors. Soluble Ep-CAM and CD-47 proteins will be produced by CHO-K1 cells transfected with cDNA encoding the extracellular region. Biotinylated antibodies will be generated using biotinylation kit (ThermoFisher, USA) by following the manufacturer's instruction.

### Binding Analysis In Vitro

The dual-antigen binding affinities of the a-CD-47/a-Ep-CAM scFv-Fc bi-specific antibody with and without mask will be evaluated by bio-layer interferometry (BLI), which is conducted on an Octet RED96 system (PALL ForteBio.). In brief, the total working volume for the samples or buffers is 0.2 mL per well, and the working temperature is set at 37° C. Briefly, streptavidin-coated biosensor tips are pre-wetted in phosphate-buffered saline (PBS), followed by the loading of the biotin-conjugated antigens Ep-CAM or CD47 (100 nM, Sino Biological Inc.). Afterwards, a series of antibody samples, including the positive controls (a-Ep-CAM mAb and a-CD47 mAb), the a-CD-47/a-Ep-CAM scFv-Fc bi-specific antibody, including masked and non-masked, are associated with the ligands in concentrations of serial dilutions. Finally, the dissociation step is conducted by dipping the sensors in PBS. Analysis is performed with Octet software, during which the association and dissociation signals are baseline corrected, and global fit is used to calculate the affinity and rate constants. Accordingly, the association rate constant (ka) indicates the Ag-Ab complex formation rate per second in a 1 M solution, and the dissociation rate constant (kd) defines the stability of the Ag-Ab complex. The affinity constant KD is calculated by the formula kd/ka [Wallner J., et al J. Pharm. Biomed. Anal. 2013; 72:150-154., Katsamba P. S., et al. Anal. Biochem. 2006; 352:208-221.].

### ADCC and CDC Lysis Assays

ADCC will be performed according to the instruction from aCella-TOX™ Bioluminescence Cytotoxicity kit (Cell Technology). In brief, human PBMC (Charls River, USA) are incubated overnight at 10≢cells/mL in IMDM human complete medium with 400 unit/mL IL-2 (PeproTech, Rocky Hill, NJ, USA). On the next day, activated PBMC are used as effector cells. 5×10\hat{ }3 HL60 cells or HT-29 cells (ATCC, USA) in 25 μL IMDM human complete medium are added per well in a U-bottom 96 well plate respectively. Then, 25 μL of IMDM human complete medium containing various antibodies at the desired concentrations is added to each well. After 5 minutes incubation at 37° C., 2.5×10\hat{ }5 PBMC in 25 μL IMDM human complete medium is added to each well to give a ratio of effector cells to target cells of 50:1. Mixtures are incubated at 37° C. for 4 hours, followed by detection using reagents supplied by the kits.

CDC assays are performed with pooled human serum collected from healthy volunteers. 5×10\hat{ }4 target cells HT29 (ATCC, USA) in 50 μL IMDM+10% FBS+Pen/Strep are added into each well of a 96-well U-bottom plate. 50 μL of antibodies, masked and non-masked a-Ep-CAM, masked and non-masked a-CD-47/a-Ep-CAM scFv-IgG1-Fc bi-specific antibody, huIgG1 isotype is added at various concentrations (diluted with medium), starting from 10 μg/mL to 10\hat{ }−5 pg/mL, with 100× dilution intervals. HL60 cell line (ATCC, USA) will be used in the assay for analysis of masked and non-masked a-CD-47 antibody, masked and non-masked a-CD-47/a-Ep-CAM scFv-hIgG1-Fc bi-specific antibody. The mixture is incubated at room temperature for 5 minutes, and then 50 μL human complement serum is added to each well, mixed, and the plate is incubated at 37° C. After 2 hours incubation, 50 μL alamar blue (AbD Serotec) is added in each well, mixed, and the plate is incubated at 37° C. overnight. On the next day, the plate is cooled to room temperature for 15 minutes and read by SpectraMax M3 plate reader for fluorescence signals at excitation wavelength of 530 nm and emission wavelength of 590 nm. Relative fluorescence unit (RFU) is used to indicate relative amount for live cells after assay.

### Phagocytosis Assay

The HL-60 cell line (ATCC, USA) is used as the target cells and labeled with CFSE and incubated with human peripheral blood-derived macrophages (Charles River, USA) in the presence of 10 μg/ml a-CD-47/a-Ep-CAM scFv-hIgG1-Fc bi-specific antibody, a-CD-47 antibody, masked a-CD-47/a-Ep-CAM scFv-hIgG1-Fc, masked a-CD-47 antibody, masked a-Ep-CAM scFv-hIgG1-Fc or isotype control. Two hours later, macrophages are imaged by fluorescence microscopy to determine the phagocytic index (number of target cells ingested per 100 macrophages). Data will be analyzed and plotted with Prism 8 (GraphPad, San Diego, USA).

In the foregoing specification, embodiments of the present invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicant to be the scope of the invention, is the literal and equivalent scope of the set of claims that issue from this application, in the specific form in which such claims issue, including any subsequent correction.

### SEQUENCE LISTING

NUMBER OF SEQ ID NOS: 70
SEQ ID NO 1
LENGTH: 162
TYPE: amino acid
ORGANISM: Homo Sapiens
FEATURE:
OTHER INFORMATION:

Sequence 1:
MRISKPHLRSISIQCYLCLLLNSHFLTEAGIHVFILGCFSAGLPKTEAN
WVNVISDLKKIEDLIQSMHIDATLYTESDVHPSCKVTAMKCFLLELQVI
SLESGDASIHDTVENLIILANNSLSSNGNVTESGCKECEELEEKNIKEF
LQSFVHIVQMFINTS

SEQ ID NO 2
LENGTH: 486
TYPE: Nucleotide
ORGANISM: Homo Sapiens
FEATURE:
OTHER INFORMATION:

Sequence 2:
ATGCGCATTAGCAAACCGCATCTGCGCAGCATTAGCATTCAGTGCTATCTGTGCCTGCT
GCTGAACAGCCATTTTCTGACCGAAGCGGGCATTCATGTGTTTATTCTGGGCTGCTTT
AGCGCGGGCCTGCCGAAAACCGAAGCGAACTGGGTGAACGTGATTAGCGATCTGAA
AAAAATTGAAGATCTGATTCAGAGCATGCATATTGATGCGACCCTGTATACCGAAAGC
GATGTGCATCCGAGCTGCAAAGTGACCGCGATGAAATGCTTTCTGCTGGAACTGCAG
GTGATTAGCCTGGAAAGCGGCGATGCGAGCATTCATGATACCGTGGAAAACCTGATTA
TTCTGGCGAACAACAGCCTGAGCAGCAACGGCAACGTGACCGAAAGCGGCTGCAAA
GAATGCGAAGAACTGGAAGAAAAAAACATTAAAGAATTTCTGCAGAGCTTTGTGCAT
ATTGTGCAGATGTTTATTAACACCAGC

SEQ ID NO 3
LENGTH: 135
TYPE: amino acid
ORGANISM: Homo sapiens
FEATURE:
OTHER INFORMATION:

Sequence 3:
MVLGTIDLCSCFSAGLPKTEANWVNVISDLKKIEDLIQSMHIDATLYTE
SDVHPSCKVTAMKCFLLELQVISLESGDASIHDTVENLIILANNSLSSN
GNVTESGCKECEELEEKNIKEFLQSFVHIVQMFINTS

SEQ ID NO 4
LENGTH: 405
TYPE: nucleotide
ORGANISM: Homo sapiens
FEATURE:
OTHER INFORMATION:

Sequence 4:
ATGGTGCTGGGCACCATTGATCTGTGCAGCTGCTTTAGCGCGGGCCTGCCGAAAACC
GAAGCGAACTGGGTGAACGTGATTAGCGATCTGAAAAAAATTGAAGATCTGATTCAG
AGCATGCATATTGATGCGACCCTGTATACCGAAAGCGATGTGCATCCGAGCTGCAAAG
TGACCGCGATGAAATGCTTTCTGCTGGAACTGCAGGTGATTAGCCTGGAAAGCGGCG
ATGCGAGCATTCATGATACCGTGGAAAACCTGATTATTCTGGCGAACAACAGCCTGAG
CAGCAACGGCAACGTGACCGAAAGCGGCTGCAAAGAATGCGAAGAACTGGAAGAA
AAAAACATTAAAGAATTTCTGCAGAGCTTTGTGCATATTGTGCAGATGTTTATTAACAC
CAGC

SEQ ID NO 5
LENGTH: 114
TYPE: amino acid
ORGANISM: Homo sapiens
FEATURE:

Sequence 5:
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS

SEQ ID NO 6
LENGTH: 342
TYPE: nucleotide
ORGANISM: Homo sapiens
FEATURE:
OTHER INFORMATION:

Sequence 6:
AACTGGGTGAACGTGATTAGCGATCTGAAAAAAATTGAAGATCTGATTCAGAGCATG
CATATTGATGCGACCCTGTATACCGAAAGCGATGTGCATCCGAGCTGCAAAGTGACCG
CGATGAAATGCTTTCTGCTGGAACTGCAGGTGATTAGCCTGGAAAGCGGCGATGCGA
GCATTCATGATACCGTGGAAAACCTGATTATTCTGGCGAACAACAGCCTGAGCAGCA
ACGGCAACGTGACCGAAAGCGGCTGCAAAGAATGCGAAGAACTGGAAGAAAAAAA
CATTAAAGAATTTCTGCAGAGCTTTGTGCATATTGTGCAGATGTTTATTAACACCAGC

SEQ ID NO 7
LENGTH: 78
TYPE: amino acid
ORGANISM: Homo Sapiens
FEATURE:
OTHER INFORMATION:

Sequence 7:
ITCPPPMSVEHADIWVKSYSLYSRERYICNSGFKRKAGTSSLTECVLNK
ATNVAHWTTPSLKCIRDPALVHQRPAPPS

SEQ ID NO 8
LENGTH: 234
TYPE: nucleotide
ORGANISM: Homo sapiens
FEATURE:
OTHER INFORMATION:

Sequence 8:
ATTACCTGCCCGCCGCCGATGAGCGTGGAACATGCGGATATTTGGGTGAAAAGCTATA
GCCTGTATAGCCGCGAACGCTATATTTGCAACAGCGGCTTTAAACGCAAAGCGGGCA
CCAGCAGCCTGACCGAATGCGTGCTGAACAAAGCGACCAACGTGGCGCATTGGACC
ACCCCGAGCCTGAAATGCATTCGCGATCCGGCGCTGGTGCATCAGCGCCCGGCGCCG
CCGAGC

SEQ ID NO 9
LENGTH: 65
TYPE: amino acid
ORGANISM: Homo Sapiens
FEATURE:
OTHER INFORMATION:

Sequence 9:
ITCPPPMSVEHADIWVKSYSLYSRERYICNSGFKRKAGTSSLTECVLNK
ATNVAHWTTPSLKCIR

SEQ ID NO 10
LENGTH: 195
TYPE: nucleotide
ORGANISM: Homo sapiens
FEATURE:
OTHER INFORMATION:

Sequence 10:
ATTACCTGCCCGCCGCCGATGAGCGTGGAACATGCGGATATTTGGGTGAAAAGCTATA
GCCTGTATAGCCGCGAACGCTATATTTGCAACAGCGGCTTTAAACGCAAAGCGGGCA
CCAGCAGCCTGACCGAATGCGTGCTGAACAAAGCGACCAACGTGGCGCATTGGACC
ACCCCGAGCCTGAAATGCATTCGC

SEQ ID NO 11
LENGTH: 92
TYPE: nucleotide
ORGANISM: synthetic construct
FEATURE:
OTHER INFORMATION:
Sequence 11:
ATGGAGTTTGGGCTGAGCTGGGTTTTCCTCGTTGCTCTTTTTAGAGGTGTCCAGTGT
GACCCAAGCTGGCTAG ATGCGCATTAGCAAACCGC
SEQ ID NO 12

Sequence 11:
ATGGAGTTTGGGCTGAGCTGGGTTTTCCTCGTTGCTCTTTTTAGAGG
TGTCCAGTGTGACCCAAGCTGGCTAG ATGCGCATTAGCAAACCGC

LENGTH: 42
TYPE: nucleotide
ORGANISM: synthetic
FEATURE:
OTHER INFORMATION:

Sequence 12:
GGTTTAAACTTAAGCTTTTA GCTGGTGTTAATAAACATCTGC

SEQ ID NO 13
LENGTH: 91
TYPE: nucleotide
ORGANISM: synthetic
FEATURE:
OTHER INFORMATION:

Sequence 13:
ATGGAGTTTGGGCTGAGCTGGGTTTTCCTCGTTGCTCTTTTTAGAGG
TGTCCAGTGTGACCCAAGCTGGCTAG ATGGTGCTGGGCACCATT

SEQ ID NO 14
LENGTH: 94
TYPE: nucleotide
ORGANISM: synthetic
FEATURE:
OTHER INFORMATION:

Sequence 14:
ATGGAGTTTGGGCTGAGCTGGGTTTTCCTCGTTGCTCTTTTTAGAGGT
GTCCAGTGTGACCCAAGCTGGCTAG AACTGGGTGAACGTGATTAGC

SEQ ID NO 15
LENGTH: 97
TYPE: nucleotide
ORGANISM: synthetic
FEATURE:
OTHER INFORMATION:

Sequence 15:
ATGAAATACCTATTGCCTACGGCAGCCGCTGGATTGTTATTACTCG
CGGCCCAGCCGGCCATGGCC GACCCAAGCTGGCTAG ATTACCTG
CCCGCCG

SEQ ID NO 16
LENGTH: 31
TYPE: nucleotide
ORGANISM: synthetic
FEATURE:
OTHER INFORMATION:

Sequence 16:
GGTTTAAACTTAAGCTTTTA CGCCGCCGAGC

SEQ ID NO 17
LENGTH: 39
TYPE: nucleotide
ORGANISM: synthetic
FEATURE:
OTHER INFORMATION:

Sequence 17:
GGTTTAAACTTAAGCTTTTA GCGAATGCATTTCAGGCTC

SEQ ID NO 18
LENGTH: 29
TYPE: nucleotide
ORGANISM: synthetic
FEATURE:
OTHER INFORMATION:

Sequence 18:
CGTGATTAGCGCGCTGAAAAAAATTGAAG

SEQ ID NO 19
LENGTH: 29
TYPE: nucleotide
ORGANISM: synthetic
FEATURE:
OTHER INFORMATION:

Sequence 19:
CTTCAATTTTTTTCAGCGCGCTAATCACG

SEQ ID NO 20
LENGTH: 23
TYPE: nucleotide
ORGANISM: synthetic
FEATURE:
OTHER INFORMATION:

Sequence 20:
TACCGTGGAAAGCCTGATTATTC

SEQ ID NO 21
LENGTH: 23
TYPE: nucleotide
ORGANISM: synthetic
FEATURE:
OTHER INFORMATION:

Sequence 21:
GAATAATCAGGCTTTCCACGGTA

SEQ ID NO 22
LENGTH: 30
TYPE: nucleotide
ORGANISM: synthetic
FEATURE:
OTHER INFORMATION:

Sequence 22:
GCATATTGTGGCGATGTTTATTAACACCAG

SEQ ID NO 23
LENGTH: 30
TYPE: nucleotide
ORGANISM: synthetic
FEATURE:
OTHER INFORMATION:

Sequence 23:
CTGGTGTTAATAAACATCGCCACAATATGC

SEQ ID NO 24
LENGTH: 29
TYPE: nucleotide
ORGANISM: synthetic
FEATURE:
OTHER INFORMATION:

Sequence 24:
CGTGATTAGCGCGCTGAAAAAAATTGAAG

SEQ ID NO 25
LENGTH: 27
TYPE: nucleotide
ORGANISM: synthetic
FEATURE:
OTHER INFORMATION:

Sequence 25:
GCCAGAATAATCAGCGCTTCCACGGTA

SEQ ID NO 26
LENGTH: 27
TYPE: nucleotide
ORGANISM: synthetic
FEATURE:
OTHER INFORMATION:

Sequence 26:
TACCGTGGAAGCGCTGATTATTCTGGC

SEQ ID NO 27
LENGTH: 30
TYPE: nucleotide
ORGANISM: synthetic
FEATURE:
OTHER INFORMATION:

Sequence 27:
CTGGTGTTAATAAACATCGCCACAATATGC

SEQ ID NO 28
LENGTH: 30
TYPE: nucleotide
ORGANISM: synthetic
FEATURE:
OTHER INFORMATION:

Sequence 28:
GCATATTGTGGCGATGTTTATTAACACCAG

SEQ ID NO 29
LENGTH: 29
TYPE: nucleotide
ORGANISM: synthetic
FEATURE:
OTHER INFORMATION:

Sequence 29:
CTTCAATTTTTTTCAGCGCGCTAATCACG

SEQ ID NO 30
LENGTH: 34
TYPE: nucleotide
ORGANISM: synthetic
FEATURE:
OTHER INFORMATION:

Sequence 30:
CGTGATTAGCAGCCTGAAAAAAATTGAAGATCTG

SEQ ID NO 31
LENGTH: 23
TYPE: nucleotide
ORGANISM: synthetic
FEATURE:
OTHER INFORMATION:

Sequence 31:
GAATAATCAGGCTTTCCACGGTA

SEQ ID NO 32
LENGTH: 23
TYPE: nucleotide
ORGANISM: synthetic
FEATURE:
OTHER INFORMATION:

Sequence 32:
TACCGTGGAAAGCCTGATTATTC

SEQ ID NO 33
LENGTH: 37
TYPE: nucleotide
ORGANISM: synthetic
FEATURE: OTHER INFORMATION:

Sequence 33:
GTTTTAGCTGGTGTTAATAAACATGCTCACAATATGC

SEQ ID NO 34
LENGTH: 37
TYPE: nucleotide
ORGANISM: synthetic
FEATURE:
OTHER INFORMATION:

Sequence 34:
GCATATTGTGAGCATGTTTATTAACACCAGCTAAAAC

SEQ ID NO 35
LENGTH: 34
TYPE: nucleotide
ORGANISM: synthetic
FEATURE:
OTHER INFORMATION:

Sequence 35:
CAGATCTTCAATTTTTTTCAGGCTGCTAATCACG

SEQ ID NO 36
LENGTH: 34
TYPE: nucleotide
ORGANISM: synthetic
FEATURE:
OTHER INFORMATION:

Sequence 36:
CGTGATTAGCAGCCTGAAAAAAATTGAAGATCTG

SEQ ID NO 37
LENGTH: 26
TYPE: nucleotide
ORGANISM: synthetic
FEATURE:
OTHER INFORMATION:

Sequence 37:
GGCTGTTGTTCGCGCTAATAATCAGG

SEQ ID NO 38
LENGTH: 26
TYPE: nucleotide
ORGANISM: synthetic
FEATURE:
OTHER INFORMATION:

Sequence 38:
CCTGATTATTAGCGCGAACAACAGCC

SEQ ID NO 39
LENGTH: 29
TYPE: nucleotide
ORGANISM: synthetic
FEATURE:
OTHER INFORMATION:

Sequence 39:
CAATATGCACAAAGCTCGCCAGAAATTCT

SEQ ID NO 40
LENGTH: 29
TYPE: nucleotide
ORGANISM: synthetic
FEATURE:
OTHER INFORMATION:

Sequence 40:
AGAATTTCTGGCGAGCTTTGTGCATATTG

SEQ ID NO 41
LENGTH: 37
TYPE: nucleotide
ORGANISM: synthetic
FEATURE:
OTHER INFORMATION:

Sequence 41:
GTTTTAGCTGGTGTTAATAAACATGCTCACAATATGC

SEQ ID NO 42
LENGTH: 36
TYPE: nucleotide
ORGANISM: synthetic
FEATURE:
OTHER INFORMATION:

Sequence 42:
GCATATTGTGAGCATGTTTATTAACACCAGCTAAAAC

SEQ ID NO 43
LENGTH: 92
TYPE: nucleotide
ORGANISM: synthetic
FEATURE:
OTHER INFORMATION:

Sequence 43:
ATGGAGTTTGGGCTGAGCTGGGTTTTCCTCGTTGCTCTTTTTAGAG
GTGTCCAGTGTGACCCAAGCTGGCTAGATGCGCATTAGCAAACCGC

SEQ ID NO 44
LENGTH: 49
TYPE: nucleotide
ORGANISM: synthetic
FEATURE:
OTHER INFORMATION:

Sequence 44:
GTGGTTGGCGCTTCTGCCGCTCAGGCTGCTGGTGTTAATAAACATCTGC

SEQ ID NO 45
LENGTH: 91
TYPE: nucleotide
ORGANISM: synthetic
FEATURE:
OTHER INFORMATION:

Sequence 45:
ATGGAGTTTGGGCTGAGCTGGGTTTTCCTCGTTGCTCTTTTTAGAGG
TGTCCAGTGTGACCCAAGCTGGCTAG ATGGTGCTGGGCACCATT

SEQ ID NO 46
LENGTH: 94
TYPE: nucleotide
ORGANISM: synthetic
FEATURE:
OTHER INFORMATION:

Sequence 46:
ATGGAGTTTGGGCTGAGCTGGGTTTTCCTCGTTGCTCTTTTTAGAGGT
GTCCAGTGTGACCCAAGCTGGCTAG AACTGGGTGAACGTGATTAGC

SEQ ID NO 47
LENGTH: 97
TYPE: nucleotide
ORGANISM: synthetic
FEATURE:
OTHER INFORMATION:

Sequence 47:
ATGAAATACCTATTGCCTACGGCAGCCGCTGGATTGTTATTACTCGCGG
CCCAGCCGGCCATGGCCGACCCAAGCTGGCTAG ATTACCTGCCCGCCG

SEQ ID NO 48
LENGTH: 44
TYPE: nucleotide
ORGANISM: synthetic
FEATURE:
OTHER INFORMATION:

Sequence 48:
GTCGCTTCTGCCGCTCAGGCCCAGAGGGCTGCCCGCCGCCGAGC

SEQ ID NO 49
LENGTH: 52
TYPE: nucleotide
ORGANISM: synthetic
FEATURE:
OTHER INFORMATION:

Sequence 49:
GTCGCTTCTGCCGCTCAGGCCCAGAGGGCTGCCGCGAATGCATT
TCAGGCTC

SEQ ID NO 50
LENGTH: 16
TYPE: nucleotide
ORGANISM: synthetic
FEATURE:
OTHER INFORMATION:

Sequence 50:
GCTAGCACCAAGGGCC

SEQ ID NO 51
LENGTH: 36
TYPE: nucleotide
ORGANISM: synthetic
FEATURE:
OTHER INFORMATION:

Sequence 51:
GGTTTAAACTTAAGCTTTTATTTACCCGGAGACAGG

SEQ ID NO 52
LENGTH: 16
TYPE: nucleotide
ORGANISM: synthetic
FEATURE:
OTHER INFORMATION:

Sequence 52:
CGTACGGTGGCTGCAC

SEQ ID NO 53
LENGTH: 39
TYPE: nucleotide
ORGANISM: synthetic
FEATURE:
OTHER INFORMATION:

Sequence 53:
GGTTTAAACTTAAGCTTCTAACACTCTCCCCTGTTGAAG

SEQ ID NO 54
LENGTH: 16
TYPE: nucleotide
ORGANISM: synthetic
FEATURE:
OTHER INFORMATION:

Sequence 54:
CAGAAGCGCCAACCAC

SEQ ID NO 55
LENGTH: 21
TYPE: nucleotide
ORGANISM: synthetic
FEATURE:
OTHER INFORMATION:

Sequence 55:
AGGCTGATCAGCGGTTTAAAC

SEQ ID NO 56
LENGTH: 16
TYPE: nucleotide
ORGANISM: synthetic
FEATURE:
OTHER INFORMATION:

Sequence 56:
GAGCGGCAGAAGCGAC

SEQ ID NO 57
LENGTH: 118
TYPE: amino acid
ORGANISM: Homo sapiens
FEATURE:
OTHER INFORMATION: heavy chain variable

Sequence 57:
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS

SEQ ID NO 58
LENGTH: 16
TYPE: nucleotide
ORGANISM: synthetic
FEATURE:
OTHER INFORMATION:

Sequence 58:
CAGAAGCGCCAACCAC

SEQ ID NO 59
LENGTH: 354
TYPE: nucleotide
ORGANISM: Homo sapiens
FEATURE:
OTHER INFORMATION: heavy chain variable

Sequence 59:
GAG GTT CAG CTC GTT GAG TCC GGC GGA GGC CTC GTA
CAG CCA GGG GGG TCT TTG CGA CTG TCC TGC GCG GCA
TCT GGG TTC ACC TTC TCC GAT AGC TGG ATA CAT TGG
GTC AGG CAA GCT CCT GGA AAA GGG CTG GAG TGG GTT
GCG TGG ATT TCC CCT TAT GGC GGG TCT ACT TAT TAT
GCG GAC AGC GTG AAG GGC AGA TTC ACC ATT TCC GCT
GAT ACT TCA AAG AAT ACA GCT TAC TTG CAG ATG AAC
TCA CTC CGC GCG GAA GAT ACT GCA GTC TAC TAC TGC
GCT CGG AGA CAT TGG CCA GGG GGG TTC GAC TAT TGG
GGC CAG GGC ACC CTG GTA ACC GTC AGT TCA

SEQ ID NO 60
LENGTH: 16
TYPE: nucleotide
ORGANISM: synthetic
FEATURE:
OTHER INFORMATION:

Sequence 60:
GCTAGCACCAAGGGCC

SEQ ID NO 61
LENGTH: 107
TYPE: amino acid
ORGANISM: Homo sapiens
FEATURE:
OTHER INFORMATION: Light chain variable

Sequence 61:
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIK

SEQ ID NO 62
LENGTH: 16
TYPE: nucleotide
ORGANISM: synthetic
FEATURE:
OTHER INFORMATION:

Sequence 62:
GAGCGGCAGAAGCGAC

SEQ ID NO 63
LENGTH: 321
TYPE: nucleotide
ORGANISM: Homo sapiens
FEATURE:
OTHER INFORMATION: light chain variable

Sequence 63:
GAT ATA CAG ATG ACG CAA TCC CCA TCC TCT TTG TCC
GCG TCC GTT GGT GAT CGG GTG ACC ATA ACC TGC CGA
GCA AGC CAA GAC GTT AGC ACG GCT GTA GCA TGG TAC
CAG CAA AAA CCA GGC AAA GCA CCA AAA CTT CTC ATA
TAC TCT GCA AGT TTT CTC TAT AGT GGA GTG CCT AGT
AGG TTC AGT GGC TCA GGC TCT GGA ACA GAC TTT ACC
CTT ACT ATC AGC TCA CTT CAG CCA GAA GAT TTC GCC
ACA TAT TAC TGC CAG CAG TAC CTT TAC CAT CCT GCG
ACT TTC GGT CAG GGG ACC AAA GTA GAA ATT AAG

SEQ ID NO 64
LENGTH: 16
TYPE: nucleotide
ORGANISM: synthetic
FEATURE:
OTHER INFORMATION:

Sequence 64:
CGTACGGTGGCTGCAC

SEQ ID NO 65
LENGTH: 117
TYPE: amino acid
ORGANISM: Homo sapiens
FEATURE:
OTHER INFORMATION: light chain variable

Sequence 65:
QVQLVQSGAEVKKPGASVKVSCKASGYTFTNYNMHWVRQAPGQRLEWMGT
IYPGNDDTSYNQKFKDRVTITADTSASTAYMELSSLRSEDTAVYYCARGG
YRAMDYWGQGTLVTVSS

SEQ ID NO 66
LENGTH: 112
TYPE: amino acid
ORGANISM: Homo Sapiens
FEATURE:
OTHER INFORMATION: Heavy chain variable

Sequence 66:
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIK

SEQ ID NO 67
LENGTH: 112
TYPE: amino acid
ORGANISM: Mus musculus
FEATURE:
OTHER INFORMATION: light chain variable

Sequence 67:
DIVMTQSALSNPVTLGESGSISCRSSKSLLHSNGITYLYWYLQKPGQSPQ
LLIYQMSNRASGVPDRFSSSGSGTDFTLKISRVEAEDVGVYYCAQNLELP
RTFGQGTKLEMK

SEQ ID NO 68
LENGTH: 116
TYPE: amino acid
ORGANISM: Mus musculus
FEATURE:
OTHER INFORMATION: Heavy chain variable

Sequence 68:
QIQLVQSGPEVKKPGESVKISCKASGYTFTKYGMNWVKQAPGQGLKWMGW
INTYTEEPTYGDDFKGRFTFTLDTSTSTAYLEISSLRSEDTATYFCARFG
SAVDYWGQGTLVTVSS

SEQ ID NO 69
LENGTH: 112
TYPE: amino acid
ORGANISM: Mus musculus
FEATURE:
OTHER INFORMATION: light chain variable

Sequence 69:
DIVMTQSALSNPVTLGESGSISCRSSKSLLHSNGITYLYWYLQKPGQSPQ
LLIYQMSNRASGVPDRFSSSGSGTDFTLKISRVEAEDVGVYYCAQNLELP
RTFGQGTKLEMK

SEQ ID NO 70
LENGTH: 116
TYPE: amino acid
ORGANISM: Mus musculus
FEATURE:
OTHER INFORMATION: Heavy chain variable

Sequence 70:
QIQLVQSGPEVKKPGESVKISCKASGYTFTKYGMNWVKQAPGQGLKWMGW
INTYTEEPTYGDDFKGRFTFTLDTSTSTAYLEISSLRSEDTATYFCARFG
SAVDYWGQGTLVTVSS

1. An antibody-based molecule or complex capable of being selectively activated in a target cell or tissue, comprising:
a first mask peptide;
a second mask peptide; and
a masked moiety comprising a full-length antibody with a specific binding ability or a fragment thereof that maintains said specific binding ability;
wherein the first mask peptide is covalently connected to the masked moiety via at least a first protease cleavable linker;
wherein the second mask peptide is covalently connected to the masked moiety via at least a second protease cleavable linker; and
wherein the first mask peptide and the second mask peptide are non-covalently bound to each other forming a hetero dimer; and said hetero dimer inhibits said specific binding ability.

2. The antibody-based molecule or complex according to claim 1, wherein the first mask peptide and the second mask peptide are, independently of each other, wildtype peptides or mutants of wildtype peptides that have no detectable biological function in vitro assay and have no biological function in vivo.

3. The antibody-based molecule or complex according to claim 1, wherein the first protease cleavable linker and the second protease cleavable linker are different or the same.

4. The antibody-based molecule or complex according to claim 1, wherein said fragment is Fab (Fragment antigen-binding), Fv (Fragment variable), F(ab′)2 (Fragment antigen-binding, 2), Fab′ (Fragment antigen-binding, prime), or scFv (Single-chain variable fragment).

5. The antibody-based molecule or complex according to claim 1, wherein said full-length antibody or said fragment thereof comprises at least one pair of light chain and heavy chain; and
wherein N terminal of the light chain is covalently connected to the first mask peptide (or the second mask peptide), and N terminal of the heavy chain is covalently connected to the second mask peptide (or the first mask peptide).

6. The antibody-based molecule or complex according to claim 1, wherein said full-length antibody or said fragment thereof comprises two pairs of light chain and heavy chain; and
wherein each of N terminals of the light chains is covalently connected to the first mask peptide (or the second mask peptide), and each of N terminals of the heavy chains is covalently connected to the second mask peptide (or the first mask peptide).

7. The antibody-based molecule or complex according to claim 1, wherein the masked moiety comprises an antibody drug conjugate, an antibody toxin conjugate, or an antibody Fc fragment fusion.

8. The antibody-based molecule or complex according to claim 1, wherein said full-length antibody or said fragment thereof is a mono clonal antibody, bi-specific antibody, an antibody domain, or a domain antibody.

9. The antibody-based molecule or complex according to claim 1, wherein the first mask peptide is a mutant (or a variant) of wildtype interleukin 15 (IL-15) and the second mask peptide is wildtype interleukin 15 receptor alpha; and
wherein the mutant (or variant) of wildtype interleukin 15 has little or no binding to the IL-15 signaling receptors including IL-2 receptor beta (CD122) and the common gamma chain (CD132)/gamma (IL-2RBY), and/or it has no detectable activity to stimulate T cell activation, as compared to wildtype IL-15 or a wildtype IL-15 receptor alpha-IL-15 fusion polypeptide.

10. The antibody-based molecule or complex according to claim 9, wherein the mutant of IL-15 comprises single mutation, double mutations, triple mutations, tetra mutations or four plus mutations.

11. The antibody-based molecule or complex according to claim 9, wherein the mutant of IL-15 may comprise substitutions of amino acid in its wildtype sequence of SEQ ID NO. 3 at the following positions: (a) D8, N65 and L69; (b) D8, N65 and Q101; (c) D8, N65 and Q108; or (d) D8, N65, Q101 and Q108; and may optionally further comprise one or more substitutions of amino acid at positions L69 and/or V104.

12. The antibody-based molecule or complex according to claim 10, wherein said substitutions of amino acid are:
D8S, D8A, or D8C;
N65A, N65Q, N65K, or N65S;
L69A, or L69R;
Q11D, Q101A, or Q101S;
V104P, or V104A; and
Q108A, Q108D, or Q108S.

13. The antibody-based molecule or complex according to claim 1, wherein said masked moiety comprises an immune checkpoint inhibitor used in cancer immunotherapy such as anti-PD-L1 antibody or Fv thereof, and wherein the antibody-based molecule or complex has significantly lower affinity to human PD-L1 or lower IL-2 production in comparison to unmasked anti-PD-L1 antibody itself or Fv thereof.

14. The antibody-based molecule or complex according to claim 1, wherein said masked moiety comprises anti-CD47 antibody, a conjugate EpCAM (Epithelial Cell Adhesion Molecule) antibody and cytotoxic drug, or a bispecific CD/47&EpCAM antibody.

15. The antibody-based molecule or complex according to claim 1, wherein the first protease cleavable linker and the second protease cleavable linker comprise, independently of each other, an amino acid sequence sensitive to one or multiple proteases; for example, they may be recognized by a serine protease, a cysteine protease, an aspartate protease, a threonine protease, a glutamic acid protease, a metalloproteinase, a gelatinase, an asparagine peptide lyase, cathepsins, a pepsin, a matriptase, a legumain, a matrix metalloprotease (MMP), a MMP1, a MMP2, a MMP3, a MMP7, a MMP8, a MMP9, a MMP10, a MMPP11, a MMP12, a MMP13, a MMP14, an ADAMS, an ADAM10, an ADAM12, an urokinase plasminogen activator (uPA), an enterokinase, a prostate-specific target (PSA, hK3), an interleukin-1β converting enzyme, a thrombin, a FAP (FAP-α), a dipeptidyl peptidase, a type II transmembrane serine protease (TTSP), a neutrophil elastase, or any combinations thereof.

16. The antibody-based molecule or complex according to claim 1, further comprising a spacer between the first mask peptide (C terminal) and the first protease cleavable linker (N terminal) and/or a spacer between the first protease cleavable linker (C terminal) and the masked moiety (N terminal), and/or
further comprising a spacer between the second mask peptide (C terminal) and the second protease cleavable linker (N terminal) and/or a spacer between the second protease cleavable linker (C terminal) and the masked moiety (N terminal); and
wherein the spacer may be for example a short amino acid sequence (e.g. 2 to 5, or 5 to 10, or 10 to 30) such as (GS)n, (GGS)n, (GGGS)n, (GGGGS)n, (SG)n, (SGG)n, (SGGG)n, (SGGGG)n, (GSGS)n (GSG)n or any other artificial or natural short amino acid sequences.

17. An expression system used for producing the antibody-based molecule or complex according to claim 1, which comprises (1) an isolated nucleic acid molecule encoding the antibody-based molecule or complex according to claim 1 or components thereof including the first mask peptide, the second mask peptide, the full-length antibody with a specific binding ability or a fragment thereof that maintains said specific binding ability, the first protease cleavable linker, and the second protease cleavable linker; (2) a vector comprising said isolated nucleic acid molecule; and/or (3) a host cell transformed with said vector.

18. A method of preparing the antibody-based molecule or complex according to claim 1, the method comprising: a) culturing a host cell comprising a vector that comprises an isolated nucleic acid molecule encoding the antibody-based molecule or complex according to claim 1; and b) recovering or harvesting the antibody-based molecule or complex from the host cell culture.

19. A pharmaceutical composition comprising (i) the antibody-based molecule or complex according to claim 1; an isolated nucleic acid molecule encoding the antibody-based molecule or complex according to claim 1; a vector comprising said isolated nucleic acid molecule; and/or a host cell transformed or transinfected with said vector; and (ii) a pharmaceutically acceptable carrier.

20. A method of treating a disease or condition including cancer, comprising administering a therapeutically effective amount of the antibody-based molecule or complex contained in or expressed by the pharmaceutical composition according to claim 19, to a subject in need thereof.


SEQUENCE LISTING (70 sequences)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - DNA, 486 bp, Homo sapiens
  source 1..486: mol_type = other DNA
atgcgcattagcaaaccgcatctgcgcagcattagcattcagtgctatctgtgcctgctgctgaacagccattttctgaccgaagcgggcattcatgtgtttattctgggctgctttagcgcgggcctgccgaaaaccgaagcgaactgggtgaacgtgattagcgatctgaaaaaaattgaagatctgattcagagcatgcatattgatgcgaccctgtataccgaaagcgatgtgcatccgagctgcaaagtgaccgcgatgaaatgctttctgctggaactgcaggtgattagcctggaaagcggcgatgcgagcattcatgataccgtggaaaacctgattattctggcgaacaacagcctgagcagcaacggcaacgtgaccgaaagcggctgcaaagaatgcgaagaactggaagaaaaaaacattaaagaatttctgcagagctttgtgcatattgtgcagatgtttattaacaccagc

SEQ ID NO: 3 - protein, 135 aa, Homo sapiens
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - DNA, 405 bp, Homo sapiens
  source 1..405: mol_type = other DNA
atggtgctgggcaccattgatctgtgcagctgctttagcgcgggcctgccgaaaaccgaagcgaactgggtgaacgtgattagcgatctgaaaaaaattgaagatctgattcagagcatgcatattgatgcgaccctgtataccgaaagcgatgtgcatccgagctgcaaagtgaccgcgatgaaatgctttctgctggaactgcaggtgattagcctggaaagcggcgatgcgagcattcatgataccgtggaaaacctgattattctggcgaacaacagcctgagcagcaacggcaacgtgaccgaaagcggctgcaaagaatgcgaagaactggaagaaaaaaacattaaagaatttctgcagagctttgtgcatattgtgcagatgtttattaacaccagc

SEQ ID NO: 5 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - DNA, 342 bp, Homo sapiens
  source 1..342: mol_type = other DNA
aactgggtgaacgtgattagcgatctgaaaaaaattgaagatctgattcagagcatgcatattgatgcgaccctgtataccgaaagcgatgtgcatccgagctgcaaagtgaccgcgatgaaatgctttctgctggaactgcaggtgattagcctggaaagcggcgatgcgagcattcatgataccgtggaaaacctgattattctggcgaacaacagcctgagcagcaacggcaacgtgaccgaaagcggctgcaaagaatgcgaagaactggaagaaaaaaacattaaagaatttctgcagagctttgtgcatattgtgcagatgtttattaacaccagc

SEQ ID NO: 7 - protein, 78 aa, Homo sapiens
ITCPPPMSVEHADIWVKSYSLYSRERYICNSGFKRKAGTSSLTECVLNKATNVAHWTTPSLKCIRDPALVHQRPAPPS

SEQ ID NO: 8 - DNA, 234 bp, Homo sapiens
  source 1..234: mol_type = other DNA
attacctgcccgccgccgatgagcgtggaacatgcggatatttgggtgaaaagctatagcctgtatagccgcgaacgctatatttgcaacagcggctttaaacgcaaagcgggcaccagcagcctgaccgaatgcgtgctgaacaaagcgaccaacgtggcgcattggaccaccccgagcctgaaatgcattcgcgatccggcgctggtgcatcagcgcccggcgccgccgagc

SEQ ID NO: 9 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 10 - DNA, 195 bp, Homo sapiens
  source 1..195: mol_type = other DNA
attacctgcccgccgccgatgagcgtggaacatgcggatatttgggtgaaaagctatagcctgtatagccgcgaacgctatatttgcaacagcggctttaaacgcaaagcgggcaccagcagcctgaccgaatgcgtgctgaacaaagcgaccaacgtggcgcattggaccaccccgagcctgaaatgcattcgc

SEQ ID NO: 11 - DNA, 92 bp, synthetic construct
  source 1..92: mol_type = other DNA
atggagtttgggctgagctgggttttcctcgttgctctttttagaggtgtccagtgtgacccaagctggctagatgcgcattagcaaaccgc

SEQ ID NO: 12 - DNA, 42 bp, synthetic construct
  source 1..42: mol_type = other DNA
ggtttaaacttaagcttttagctggtgttaataaacatctgc

SEQ ID NO: 13 - DNA, 91 bp, synthetic construct
  source 1..91: mol_type = other DNA
atggagtttgggctgagctgggttttcctcgttgctctttttagaggtgtccagtgtgacccaagctggctagatggtgctgggcaccatt

SEQ ID NO: 14 - DNA, 94 bp, synthetic construct
  source 1..94: mol_type = other DNA
atggagtttgggctgagctgggttttcctcgttgctctttttagaggtgtccagtgtgacccaagctggctagaactgggtgaacgtgattagc

SEQ ID NO: 15 - DNA, 97 bp, synthetic construct
  source 1..97: mol_type = other DNA
atgaaatacctattgcctacggcagccgctggattgttattactcgcggcccagccggccatggccgacccaagctggctagattacctgcccgccg

SEQ ID NO: 16 - DNA, 31 bp, synthetic construct
  source 1..31: mol_type = other DNA
ggtttaaacttaagcttttacgccgccgagc

SEQ ID NO: 17 - DNA, 39 bp, synthetic construct
  source 1..39: mol_type = other DNA
ggtttaaacttaagcttttagcgaatgcatttcaggctc

SEQ ID NO: 18 - DNA, 29 bp, synthetic construct
  source 1..29: mol_type = other DNA
cgtgattagcgcgctgaaaaaaattgaag

SEQ ID NO: 19 - DNA, 29 bp, synthetic construct
  source 1..29: mol_type = other DNA
cttcaatttttttcagcgcgctaatcacg

SEQ ID NO: 20 - DNA, 23 bp, synthetic construct
  source 1..23: mol_type = other DNA
taccgtggaaagcctgattattc

SEQ ID NO: 21 - DNA, 23 bp, synthetic construct
  source 1..23: mol_type = other DNA
gaataatcaggctttccacggta

SEQ ID NO: 22 - DNA, 30 bp, synthetic construct
  source 1..30: mol_type = other DNA
gcatattgtggcgatgtttattaacaccag

SEQ ID NO: 23 - DNA, 30 bp, synthetic construct
  source 1..30: mol_type = other DNA
ctggtgttaataaacatcgccacaatatgc

SEQ ID NO: 24 - DNA, 29 bp, synthetic construct
  source 1..29: mol_type = other DNA
cgtgattagcgcgctgaaaaaaattgaag

SEQ ID NO: 25 - DNA, 27 bp, synthetic construct
  source 1..27: mol_type = other DNA
gccagaataatcagcgcttccacggta

SEQ ID NO: 26 - DNA, 27 bp, synthetic construct
  source 1..27: mol_type = other DNA
taccgtggaagcgctgattattctggc

SEQ ID NO: 27 - DNA, 30 bp, synthetic construct; identical to SEQ ID NO: 23
  source 1..30: mol_type = other DNA

SEQ ID NO: 28 - DNA, 30 bp, synthetic construct; identical to SEQ ID NO: 22
  source 1..30: mol_type = other DNA

SEQ ID NO: 29 - DNA, 29 bp, synthetic construct
  source 1..29: mol_type = other DNA
cttcaatttttttcagcgcgctaatcacg

SEQ ID NO: 30 - DNA, 34 bp, synthetic construct
  source 1..34: mol_type = other DNA
cgtgattagcagcctgaaaaaaattgaagatctg

SEQ ID NO: 31 - DNA, 23 bp, synthetic construct
  source 1..23: mol_type = other DNA
gaataatcaggctttccacggta

SEQ ID NO: 32 - DNA, 23 bp, synthetic construct
  source 1..23: mol_type = other DNA
taccgtggaaagcctgattattc

SEQ ID NO: 33 - DNA, 37 bp, synthetic construct
  source 1..37: mol_type = other DNA
gttttagctggtgttaataaacatgctcacaatatgc

SEQ ID NO: 34 - DNA, 37 bp, synthetic construct
  source 1..37: mol_type = other DNA
gcatattgtgagcatgtttattaacaccagctaaaac

SEQ ID NO: 35 - DNA, 34 bp, synthetic construct
  source 1..34: mol_type = other DNA
cagatcttcaatttttttcaggctgctaatcacg

SEQ ID NO: 36 - DNA, 34 bp, synthetic construct; identical to SEQ ID NO: 30
  source 1..34: mol_type = other DNA

SEQ ID NO: 37 - DNA, 26 bp, synthetic construct
  source 1..26: mol_type = other DNA
ggctgttgttcgcgctaataatcagg

SEQ ID NO: 38 - DNA, 26 bp, synthetic construct
  source 1..26: mol_type = other DNA
cctgattattagcgcgaacaacagcc

SEQ ID NO: 39 - DNA, 29 bp, synthetic construct
  source 1..29: mol_type = other DNA
caatatgcacaaagctcgccagaaattct

SEQ ID NO: 40 - DNA, 29 bp, synthetic construct
  source 1..29: mol_type = other DNA
agaatttctggcgagctttgtgcatattg

SEQ ID NO: 41 - DNA, 37 bp, synthetic construct; identical to SEQ ID NO: 33
  source 1..37: mol_type = other DNA

SEQ ID NO: 42 - DNA, 37 bp, synthetic construct; identical to SEQ ID NO: 34
  source 1..37: mol_type = other DNA

SEQ ID NO: 43 - DNA, 92 bp, synthetic construct; identical to SEQ ID NO: 11
  source 1..92: mol_type = other DNA

SEQ ID NO: 44 - DNA, 49 bp, synthetic construct
  source 1..49: mol_type = other DNA
gtggttggcgcttctgccgctcaggctgctggtgttaataaacatctgc

SEQ ID NO: 45 - DNA, 91 bp, synthetic construct; identical to SEQ ID NO: 13
  source 1..91: mol_type = other DNA

SEQ ID NO: 46 - DNA, 94 bp, synthetic construct; identical to SEQ ID NO: 14
  source 1..94: mol_type = other DNA

SEQ ID NO: 47 - DNA, 97 bp, synthetic construct; identical to SEQ ID NO: 15
  source 1..97: mol_type = other DNA

SEQ ID NO: 48 - DNA, 44 bp, synthetic construct
  source 1..44: mol_type = other DNA
gtcgcttctgccgctcaggcccagagggctgcccgccgccgagc

SEQ ID NO: 49 - DNA, 52 bp, synthetic construct
  source 1..52: mol_type = other DNA
gtcgcttctgccgctcaggcccagagggctgccgcgaatgcatttcaggctc

SEQ ID NO: 50 - DNA, 16 bp, synthetic construct
  source 1..16: mol_type = other DNA
gctagcaccaagggcc

SEQ ID NO: 51 - DNA, 36 bp, synthetic construct
  source 1..36: mol_type = other DNA
ggtttaaacttaagcttttatttacccggagacagg

SEQ ID NO: 52 - DNA, 16 bp, synthetic construct
  source 1..16: mol_type = other DNA
cgtacggtggctgcac

SEQ ID NO: 53 - DNA, 39 bp, synthetic construct
  source 1..39: mol_type = other DNA
ggtttaaacttaagcttctaacactctcccctgttgaag

SEQ ID NO: 54 - DNA, 16 bp, synthetic construct
  source 1..16: mol_type = other DNA
cagaagcgccaaccac

SEQ ID NO: 55 - DNA, 21 bp, synthetic construct
  source 1..21: mol_type = other DNA
aggctgatcagcggtttaaac

SEQ ID NO: 56 - DNA, 16 bp, synthetic construct
  source 1..16: mol_type = other DNA
gagcggcagaagcgac

SEQ ID NO: 57 - protein, 118 aa, Homo sapiens [sequence omitted]

SEQ ID NO: 58 - DNA, 16 bp, synthetic construct
  source 1..16: mol_type = other DNA
cagaagcgccaaccac

SEQ ID NO: 59 - DNA, 354 bp, Homo sapiens
  source 1..354: mol_type = other DNA; note = heavy chain variable
gaggttcagctcgttgagtccggcggaggcctcgtacagccaggggggtctttgcgactgtcctgcgcggcatctgggttcaccttctccgatagctggatacattgggtcaggcaagctcctggaaaagggctggagtgggttgcgtggatttccccttatggcgggtctacttattatgcggacagcgtgaagggcagattcaccatttccgctgatacttcaaagaatacagcttacttgcagatgaactcactccgcgcggaagatactgcagtctactactgcgctcggagacattggccaggggggttcgactattggggccagggcaccctggtaaccgtcagttca

SEQ ID NO: 60 - DNA, 16 bp, synthetic construct
  source 1..16: mol_type = other DNA
gctagcaccaagggcc

SEQ ID NO: 61 - protein, 107 aa, Homo sapiens [sequence omitted]

SEQ ID NO: 62 - DNA, 16 bp, synthetic construct
  source 1..16: mol_type = other DNA
gagcggcagaagcgac

SEQ ID NO: 63 - DNA, 321 bp, Homo sapiens
  source 1..321: mol_type = other DNA; note = light chain variable
gatatacagatgacgcaatccccatcctctttgtccgcgtccgttggtgatcgggtgaccataacctgccgagcaagccaagacgttagcacggctgtagcatggtaccagcaaaaaccaggcaaagcaccaaaacttctcatatactctgcaagttttctctatagtggagtgcctagtaggttcagtggctcaggctctggaacagactttacccttactatcagctcacttcagccagaagatttcgccacatattactgccagcagtacctttaccatcctgcgactttcggtcaggggaccaaagtagaaattaag

SEQ ID NO: 64 - DNA, 16 bp, synthetic construct
  source 1..16: mol_type = other DNA
cgtacggtggctgcac

SEQ ID NO: 65 - protein, 117 aa, Homo sapiens
  source 1..117: note = light chain variable
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 66 - protein, 112 aa, Homo sapiens
  source 1..112: note = Heavy chain variable
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 67 - protein, 112 aa, Mus musculus
  source 1..112: note = light chain variable
DIVMTQSALSNPVTLGESGSISCRSSKSLLHSNGITYLYWYLQKPGQSPQLLIYQMSNRASGVPDRFSSSGSGTDFTLKISRVEAEDVGVYYCAQNLELPRTFGQGTKLEMK

SEQ ID NO: 68 - protein, 116 aa, Mus musculus
  source 1..116: note = Heavy chain variable
QIQLVQSGPEVKKPGESVKISCKASGYTFTKYGMNWVKQAPGQGLKWMGWINTYTEEPTYGDDFKGRFTFTLDTSTSTAYLEISSLRSEDTATYFCARFGSAVDYWGQGTLVTVSS

SEQ ID NO: 69 - protein, 112 aa, Mus musculus; identical to SEQ ID NO: 67
  source 1..112: note = light chain variable

SEQ ID NO: 70 - protein, 116 aa, Mus musculus; identical to SEQ ID NO: 68
  source 1..116: note = Heavy chain variable